(19) United States
(12) Patent Application Publication
(10) Pub. No.: US 2010/0267122 A1
(43) Pub. Date: Oct. 21, 2010

(54) MICROALGAE CULTIVATION IN A WASTEWATER DOMINATED BY CARPET MILL EFFLUENTS FOR BIOFUEL APPLICATIONS

(76) Inventors: Senthil Chinnasamy, Athens, GA (US); Ashish Bhatnagar, Ajmer (IN); Ryan W. Hunt, Athens, GA (US); Ronald Claxton, Athens, GA (US); Mark Marlowe, Dalton, GA (US); Keshav C. Das, Athens, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/756,371

(22) Filed: Apr. 8, 2010

Related U.S. Application Data
(60) Provisional application No. 61/170,164, filed on Apr. 17, 2009.

(51) Int. Cl. C12N 1/12

(52) U.S. Cl. 435/257.3; 435/257.1; 435/257.5

(57) ABSTRACT

The disclosure encompasses, among other aspects, mixed algal populations able to survive and proliferate on culture media that have a high proportion of carpet industry wastewater. Embodiments further encompass methods of cultivating mixed populations of freshwater and marine alga comprising a plurality of genera and species to provide a biomass from which may be extracted lipids, or converted into biodiesel by such procedures as pyrolysis. Lipid material extracted from the algae may be converted to biodiesel or other organic products. A combined stream of carpet industry untreated wastewater with 10-15% sewage was found to be a good growth medium for cultivation of microalgae and biodiesel production. Native algal strains were isolated from carpet wastewater inoculated with mixed populations derived from environments exposed to such wastewater. Both fresh water and marine algae showed good growth in wastewaters. About 65% of the algal oil obtained from the algal consortium cultured on carpet industry wastewater could be converted into biodiesel.

### CROSS-REFERENCE TO RELATED APPLICATIONS

This application claims priority to U.S. Provisional Patent Application Ser. No. 61/170,164, entitled “RENEWABLE BIOMASS, BIOFUEL AND BIOPRODUCTION FROM CARPET INDUSTRY WASTEWATER (TREATED AND UNTREATED) USING MIXOTROPHIC ALGA(E)” filed on Apr. 17, 2009, the entirety of which is hereby incorporated by reference.

### TECHNICAL FIELD

The present disclosure is generally related to mixed algal compositions able to proliferate on carpet industry wastewater, and to methods of obtaining an algal biomass from such cultures for use in generating a biofuel.

### BACKGROUND

Various estimates support that apart from drinking water, farmers will need about 4000 cubic kilometers of water in 2050, as against the current 2700 cubic kilometers, if no new technological changes are deployed to reduce water usage (Amarsinghe et al., (2007) IWMI Research Report 123). Of the estimated water use, the global target for biofuel feedstock crop production for 2030 itself would demand 180 cubic kilometers of water (IWMI, (2008) Water Policy Brief, Issue 30). Algae are considered an economically viable alternative to present biofuel crops such as corn and soybean as they do not require arable land (Chisti Y. (2007) Biotechnol. Adv. 25: 294-306; Hu et al., (2008) Plant J. 54: 621-639). However, their water demand is as high as 11-13 million liters per hectare for cultivation in open ponds. Their capability to grow in industrial, municipal and agricultural wastewaters and seawater cannot only overcome this hurdle, but also can simultaneously provide treated water suitable for other uses. Oswald, as early as 1963 (Dev. Ind. Microbiol. 4: 112-119) honed this process of phycoremediation of wastewaters and suggested a number of byproduct applications for the biomass generated.

Besides agricultural use of water, mainly as irrigation, annual water use for domestic purposes between 1987 and 2003 was estimated as about 325 billion cubic meters. Industries consumed 665 billion cubic meters water annually during the same period. Most wastewater is polluting and creating health hazards. If 50% of this non-agricultural consumed water is available for algae production, it would have the potential to generate up to about 250 million tons of algal biomass, including 37 million tons of oil. However, variations in the compositions of wastewaters limit those algae species that may be useful for cultivation on wastewater.

In one area of the U.S. having a concentration of carpet-producing industries, North Central Georgia, wastewater generated by carpet mills along with city sewage (combined volume of between 40-55 million cubic meters per annum) has the potential to generate up to 15,000 tons of algal biomass, which could provide between about 2.5 and about 4 million liters of biodiesel, and remove up to about 1500 tons of nitrogen and about 150 tons of phosphorus from the wastewater per year.

The carpet industry in the US must meet stringent requirements on the quality of wastewater it discharges from carpet manufacturing plants. Current waste treatment procedures are expensive and the industry is interested in reducing cost of waste management. The focus on wastewater treatment has shifted from pollution control to resource exploitation in view of technical feasibility, economics, societal needs and political priorities (Argenent et al., 2004). Many bioprocesses can provide bioenergy while simultaneously achieving the objective of pollution control, which could reduce the cost of wastewater treatment, and reduce dependence of fossil fuels.

### SUMMARY

One aspect of the present disclosure encompasses methods of generating an algal biomass, comprising: (a) forming an algal culture by combining: (i) a population of algal cells characterized as proliferating in a medium comprising carpet industry wastewater, and (ii) a culture medium comprising carpet industry wastewater; and (b) maintaining the algal culture under conditions suitable for the proliferation of the population of algal cells, thereby forming an algal biomass. In embodiments of this aspect of the disclosure, the medium may further comprise a sewage system effluent.

In the embodiments of the methods of this aspect of the disclosure, the population of algal cells can comprise at least one of the group consisting of: a marine algal strain, a freshwater (non-marine) algal strain, a cyanobacter strain, a diatomaceous algal strain, a plurality of marine algal strains, a plurality of freshwater (non-marine) algal strains, a plurality of cyanobacter strains, and a plurality of diatomaceous algal strains, or any combination thereof, and at least one algal strain of the population of algal cells can be isolated from a source in contact with the wastewater effluent of the carpet industry.

Another aspect of the disclosure encompasses methods of producing a biofuel from carpet industry wastewater comprising: (a) forming an algal culture by combining: (i) a population of algal cells characterized as proliferating in a medium comprising carpet industry wastewater, and (ii) a culture medium comprising carpet industry wastewater; (b) maintaining the algal culture under conditions suitable for proliferation of the population of algal cells, thereby forming an algal biomass; (c) isolating the algal biomass from the medium; and (d) obtaining from the isolated algal biomass a biofuel or a lipid material convertible to a biofuel.

Still another aspect of the disclosure encompasses a system for generating an algal biomass, the system comprising an algal culture container selected from a raceway, a vertical tower reactor, a polybag, or a plurality of any thereof, and where the container or plurality of containers is optionally provided with an air supply supplemented with carbon dioxide; an algal culture medium comprising carpet industry wastewater and optionally a sewage system effluent; and a population of algal cells in the algal culture medium, where the algal cells can be selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, a Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

Yet another aspect of the disclosure comprises embodiments of an isolated population of algal cells comprising at least one algal strain isolated from a source in contact with the wastewater effluent of the carpet industry and capable of proliferating on a medium comprising carpet industry wastewater.

### BRIEF DESCRIPTION OF THE DRAWINGS

Further aspects of the present disclosure will be more readily appreciated upon review of the detailed description of its various embodiments, described below, when taken in conjunction with the accompanying figures.

FIG. 1 shows a graph illustrating the growth responses of various strains of microalgae in carpet industry treated and untreated wastewater, and standard growth medium. 1, Botryococcus braunii UTEX 572; 2, Chlorella protothecoides UTEX 25; 3, C. saccharophila var. saccharophila UTEX 2469; 4, C. vulgaris UTEX 2714; 5, Cricosphaera carterae UTEX LB1014; 6, Dunaliella tertiolecta UTEX LB999; 7, Nannochloris oculata UTEX LB1998; 8, Spirulina platensis UTEX LB1926; 9, S. maxima UTEX LB2342; 10, Tetraselmis suecica UTEX LB2286; 11, T. chuii UTEX LB232; 12, Phaeodactylum tricornutum UTEX 646; 13, Pleurochtysis carterae CCMP 647; and 14, Consortium.

FIG. 2 shows a graph illustrating the biomass production potential of the algal consortium at 25° C. and 15° C., and at ambient (0.03%) and elevated (6%) CO₂ levels.

FIG. 3 shows a schematic for biofuel production using carpet industry wastewater.

FIG. 4A is a graph illustrating the productivity of algae consortium with respect to changes in temperature in raceways, vertical tube reactors, and polybags.

FIG. 4B is a graph illustrating the productivity of algae consortium with respect to changes in pH in raceways, vertical tube reactors and polybags.

FIG. 4C is a graph illustrating the productivity of algae consortium with respect to changes in light penetration depth in raceways, vertical tube reactors and polybags.

FIG. 5 schematically illustrates a variety of polybag arrangements for attaining maximum biomass productivity.

FIG. 6 is a graph illustrating the estimated algal biomass production potential of raceways, vertical tank reactors (vtr) and polybags based on the 22 year irradiance data of a city in North Georgia, U.S.A. and biomass productivity of algal consortium in carpet industry (CI) untreated wastewater.

FIG. 7 is a bar chart illustrating the composition of algal biomass consortium grown in untreated carpet industry wastewater. Results indicated that the microalgae consortium was rich in proteins and low in carbohydrates and lipids.

The details of some exemplary embodiments of the methods and systems of the present disclosure are set forth in the description below. Other features, objects, and advantages of the disclosure will be apparent to one of skill in the art upon examination of the following description, drawings, examples and embodiments. It is intended that all such additional systems, methods, features, and advantages be included within this description, be within the scope of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### DEFINITIONS

In describing the disclosed subject matter, the following terminology will be used in accordance with the definitions set forth below.

The term “wastewater” as used herein refers to the effluent from a manufacturing plant for the production of carpet. Typically, such wastewater comprises the chemical components resulting from the preparation of yarn or other materials used in the fabrication of a carpet including, but not limited to, organic substances derived from such as the fibrous material of a carpet, raw materials thereof, metal ions, acids, alkalis, salts, dye components and the like. Carpet industry wastewater may further comprise solutions or suspensions of compounds or particles from the carpet manufacturing process.

The term “untreated wastewater” as used herein refers to water effluent directly from a carpet manufacturing plant without removal of any materials used in, or resulting from, the manufacturing process. The “untreated wastewater” may then be supplemented with effluent from a municipal sewage system that includes in varying amounts residential and commercial sewage.

The term “treated wastewater” as used herein refers to effluent wastewater from a carpet manufacturing facility that has been combined with an amount of a municipal (residential and commercial) sewage and which has been processed in a sewage or water treatment plant such as by an activated sludge process for the removal or reduction in the level of the carbon and biological loads, metals, etc. Typically, the treated wastewater can be contained within a reservoir open to the atmosphere before disposal such as by spraying onto to land surfaces for further treatment, and while rendered suitable for adding to general sewage or land disposal may include dye components, organic material and the like that can support the growth of microorganisms, including algae.

The term “algae” as used herein refers to any organisms with chlorophyll and, in other than unicellular algae, a thallus not differentiated into roots, stems and leaves, and encompasses prokaryotic and eukaryotic organisms that are photoautotrophic or facultative heterotrophs. The term “algae” includes macroalgae (such as seaweed) and microalgae. For certain embodiments of the disclosure, algae that are not macroalgae are preferred. The terms “microalgae” and “phytoplankton,” used interchangeably herein, refer to any microscopic algae, photoautotrophic or facultative heterotroph protozoa, photoautotrophic or facultative heterotroph prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae.

The algae of the disclosure can be a naturally occurring species, a genetically selected strain, a genetically manipulated strain, a transgenic strain, or a synthetic alga. Algae from tropical, subtropical, temperate, polar or other climatic regions can be used in the disclosure. Endemic or indigenous algal species are generally preferred over introduced species where an open culturing system is used. Algae, including microalgae, inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Any of such aquatic environments, freshwater species, marine species, and/or species that thrive in varying and/or intermediate salinities or nutrient levels, can be used in the embodiments of the disclosure. The algae in an algal composition of the disclosure may contain a mixture of prokaryotic and eukaryotic organisms, wherein some of the species may be unidentified. Fresh water from rivers, lakes; seawater from coastal areas, oceans; water in hot springs or thermal vents; and lake, marine, or estuarine sediments, can be used to source the algae. The algae may also be collected from local or remote bodies of water, including surface as well as subterranean water. Preferably, the algal species for use in the embodiments of the disclosure may be isolated from water or soil that has been in contact with high volumes of carpet industry wastewater for a prolonged period. This period of exposure will advantageously enrich the population of algae proliferating therein in those species and strains of algae able to utilize the wastewater as a nutrient source. It is not required that all the algae in an algal composition of the disclosure be taxonomically classified or characterized for the composition be used in the present disclosure. Algal compositions including algal cultures can be distinguished by the relative proportions of taxonomic groups that are present.

One or more species of algae are present in the algal composition of the disclosure. In one embodiment of the disclosure, the algal composition is a monoculture, wherein only one species of algae is grown. However, in many open culturing systems, it may be difficult to avoid the presence of other algae species in the medium. Accordingly, a monoculture may comprise about 0.1% to 2% cells of algae species other than the intended species, i.e., up to 98% to 99.9% of the algal cells in a monoculture are of one species. In certain embodiments, the algal compositions comprise an isolated species of algae, such as an axenic culture. In other embodiments, the algal composition can be a mixed culture that comprises more than one species of algae, i.e., the algal culture is not a monoculture. Such a culture can occur naturally with an assemblage of different species of algae or it can be prepared by mixing different algal cultures or axenic cultures. In certain embodiments, an algal composition comprising a combination of different batches of algal cultures is used in the disclosure. The algal composition can be prepared by mixing a plurality of different algal cultures. The different taxonomic groups of algae can be present in defined proportions. The combination and proportion of different algae in an algal composition can be designed or adjusted to yield a desired blend of algal lipids.

A mixed algal composition of the disclosure comprises one or several dominant species of macroalgae and/or microalgae. Microalgal species can be identified by microscopy and enumerated by counting, by microfluidics, or by flow cytometry, which are techniques well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98% of the algae present in the culture. In certain mixed algal composition, several dominant algae species may each independently constitute greater than about 10%, about 20%, about 30%, about 40%, about 50%, about 60%, about 70%, about 80% or about 90% of the algae present in the culture. Many other minor species of algae may also be present in such compositions but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. It is to be understood that mixed algal cultures or compositions having the same genus or species of algae may be different by virtue of the relative abundance of the various genus and/or species that are present. It is understood that for the purposes of the embodiments of the disclosure, the populations of algae, either monoculture or mixed populations are characterized as being able to proliferate on a medium comprising carpet industry wastewater, either untreated or treated to further comprise an amount of city sewage that allows growth of the algae to preferably increase over the growth rate in the absence of the added sewage. It is further understood that with a mixed population of algae, two or more of the species or strains of the mixed population may differ in their growth rates when cultured on carpet industry wastewater-based media.

It should also be understood that in certain embodiments, such algae may be present as a contaminant, a non-dominant group or a minor species, especially in an open system. Such algae may be present in negligent numbers, or substantially diluted given the volume of the culture or composition. The presence of such algal genus or species in a culture, composition or a body of water is distinguishable from cultures, composition or bodies of water where such algal genus or species are dominant, or constitute the bulk of the algae. In various embodiments, one or more species of algae belonging to the following phyla can be used in the systems and methods of the disclosure: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds and/or macroalgae, many of which belong to the phyla Phaeophyta or Rhodophyta, are less preferred.

In certain embodiments, the algal composition of the disclosure comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal composition of the disclosure comprises algae from one or more of the following taxonomic classes: Euglenophyceae, Dinophyceae, and Ebriophyceae. Non-limiting examples include Euglena species and the freshwater or marine dinoflagellates.

In certain embodiments, the algal composition of the disclosure comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii).

In certain embodiments, the algal composition of the disclosure comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal composition in the disclosure comprises freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In certain embodiments, the algal composition of the disclosure comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In certain embodiments, any of the above-mentioned genus and species of algae may each be less preferred independently as a dominant species in, or be excluded from, an algal composition of the disclosure

The term “consortium” as used herein refers to a population of a plurality of algal species that are able to survive and proliferate using a culture medium, the culture medium comprising a treated or untreated wastewater effluent from a carpet manufacturing plant combined with municipal commercial and residential sewage. The “consortium” may be assembled from isolates of algal species or isolated as a group of algal strains from a natural environment such as, but not limited to, a wastewater holding reservoir. In such a case as a holding reservoir, it is contemplated that the isolated algal strains will be able to proliferate on the wastewater, although increases in their growth rates and biomass yields may be increased by subsequent genetic modification of by additions or modifications to the culture medium. The term “primary consortium” as used herein refers to a population of about 15 algal strains initially isolated from a medium enriched in carpet industry wastewater ad inoculated with isolates from a storage pond or a location subject to prolonged exposure to carpet industry wastewater. Most advantageously for use in the methods of the disclosure the consortium comprised three strains of algae: Chlamydomonas globosa, Chlorella minutissima, and Scenedesmus bijuga, that were isolated as the predominant strains of a culture of the primary consortium after growth on the carpet industry wastewater-based medium.

Any named herein as being adapted for growth in the carpet industry wastewater will be suitable for use in the aquaculture system and method of the disclosure. Exemplary species include, by way of example and without limitation, microalgae such as Porphyridium cruentum, Spirulina platensis, Cyclotella nana, Dunaliella salina, Dunaliella bardawil, Phaeodactylum tricornutum, Muriellopsis spp., Chlorella fusca, Chlorella zofingiensis, Chlorella spp., Haematococcus pluvialis, Chlorococcum citriforme, Neospongiococcum gelatinosum, Isochrysis galbana, Chlorella stigmataphora, Chlorella vulgaris, Chlorella pyrenoidosa, Chlamydomonas mexicana, Scenedesmus obliquus, Scenedesmus braziliensis, Stichococcus bacillaris, Anabaena flos-aquae, Porphyridium aerugineum, Fragilaria sublinearis, Skeletonema costatum, Pavlova gyrens, Monochrysis lutheri, Coccolithus huxleyi, Nitzschia palea, Dunaliella tertiolecta, Prymnesium paruum, and the like.

The term “photoautotroph” as used herein refers to organisms (usually plants) that carry out photosynthesis to acquire energy. Energy from sunlight is used to convert carbon dioxide and water into organic materials to be used in cellular functions such as biosynthesis and respiration. In an ecological context, they provide nutrition for all other forms of life (besides other autotrophs such as chemotrophs). In terrestrial environments, plants are the predominant variety, while aquatic environments include a range of phototrophic organisms such as algae (e.g. kelp), other protists (such as euglena) and bacteria (such as cyanobacteria). One product of this process is starch, which is a storage or reserve form of carbon, which can be used when light conditions are too poor to satisfy the immediate needs of the organism. Photosynthetic bacteria have a substance called bacteriochlorophyll, live in lakes and pools, and use the hydrogen from hydrogen sulfide instead of from water, for the chemical process. Cyanobacteria live in fresh water, seas, soil and lichen, and use a plant-like photosynthesis. The depth to which sunlight or artificial light can penetrate into water, so that photosynthesis may occur, is known as the phototrophic zone.

The term “autotroph” as used herein refers to an organism that produces complex organic compounds (carbohydrates, fats, and proteins) from simple inorganic molecules using energy from light (by photosynthesis) or inorganic chemical reactions. They are able to make their own food and can convert carbon dioxide into useful, solid compounds (such as long chain carbon compounds necessary for growth). Therefore, they do not utilize organic compounds as an energy source or a carbon source. Through reduction (a form of chemical reaction where hydrogen is added to the chemical chain), autotrophs can reduce carbon dioxide to organic compounds. The reduction of carbon dioxide, a low-energy compound, creates a store of chemical energy. Most autotrophs use water as the reducing agent, but some can use other hydrogen compounds such as hydrogen sulfide. Autotrophs are the producers in a food chain, such as plants on land or algae in water. Bacteria which derive energy from oxidizing inorganic compounds (such as hydrogen sulfide, elemental sulfur, ammonium and ferrous iron) are chemoautotrophs, and include the lithotrophs.

The term “heterotroph” as used herein refers to an organism that uses organic carbon for growth. This contrasts with autotrophs, such as plants, which can directly use sources of energy such as light to produce organic substrates from inorganic carbon dioxide.

The term “biomass” as utilized herein refers to the mass or accumulating mass of photosynthetic organisms resulting from the cultivation of such organisms using a variety of techniques.

The terms “photobioreactor,” “photobioreactor apparatus”, or “reactor” as used herein refer to an apparatus containing, or configured to contain, a liquid medium comprising at least one species of photosynthetic organism and having either a source of light capable of driving photosynthesis associated therewith, or having at least one surface at least a portion of which is partially transparent to light of a wavelength capable of driving photosynthesis (i.e. light of a wavelength between about 400-700 nm). Certain photobioreactors for use herein comprise an enclosed bioreactor system such as, but not limited to, a polybag, as contrasted with an open bioreactor, such as a pond or other open body of water, open tanks, open channels such as a raceway, and the like.

The term “raceway” as used herein refers toelongated (long and narrow) tanks or liquid paths that provide a flow-through system for a culture medium, thereby enabling a higher yield of biomass than would be achieved by a static pond system.

The term “biofuel” as used herein refers to fuel derived from biomass. The term “biomass” encompasses solid biomass, liquid fuels and various biogases. Bioethanol is an alcohol made by fermenting the sugar components of plant materials and it is made mostly from sugar and starch crops. With advanced technology being developed, cellulosic biomass, such as trees and grasses, are also used as feedstocks for ethanol production. Ethanol can be used as a fuel for vehicles in its pure form, but it is usually used as a gasoline additive. The predominant biogas produced from a biomass is typically methane but may also include minor percentages of other alkyl-chain gases and volatile compounds.

The term “biodiesel” as used herein refers to a vegetable oil- or animal fat-based diesel fuel consisting of long-chain alkyl (methyl, propyl or ethyl) esters. Biodiesel is typically made by chemically reacting lipids, such as derived from algae cultured by the methods of the present disclosure, with an alcohol. Biodiesel is produced from oils or fats using transesterification. Biodiesel is meant to be used in standard diesel engines and is distinct from the vegetable and waste oils. Biodiesel can be used alone, or blended with petrodiesel. The term “biodiesel” can be standardized as mono-alkyl ester in the United States.

Generally, a process for production of biofuels from algae can include cultivating an oil-producing algae by promoting both autotrophic and heterotrophic growth. Heterotrophic growth can include introducing an algal feed to the oil-producing algae to increase the formation of algal oil. The algal oil can be extracted from the oil-producing algae using biological agents and/or other methods such as mechanical pressing. The resulting algal oil can be subjected to a transesterification process to form biodiesel.

The terms “transesterify,” “transesterifying,” and “transesterification” refer to a process of exchanging an alkoxy group of an ester by another alcohol and more specifically, of converting algal oil, e.g. triglycerides, to biodiesel, e.g. fatty acid alkyl esters, and glycerol. Transesterification can be accomplished by using traditional chemical processes such as acid or base catalyzed reactions, or by using enzyme-catalyzed reactions.

### Discussion

The embodiments of the present disclosure encompass, among other aspects, mixed algal populations able to survive and proliferate on culture media that have a high proportion of carpet industry wastewater. The embodiments of the disclosure further encompass methods of cultivating mixed populations of freshwater and marine alga comprising a plurality of genera and species to provide a biomass from which may be extracted lipids, or converted into biodiesel by such procedures as pyrolysis. Lipid material extracted from the algae may be converted to biodiesel or other organic products.

Carpet mill wastewaters show wide variation in quality. A stream of carpet industry untreated wastewater combined with 10-15% sewage has been found to be a good growth medium for cultivation of microalgae. Algal biomass and biodiesel production using a wastewater containing 85-90% carpet industry effluents treated with 10-15% municipal sewage was shown. Native algal strains were isolated from carpet wastewater inoculated with mixed populations derived from environments exposed to such wastewater. Growth studies indicated both fresh water and marine algae showed good growth in wastewaters. A consortium of native algal isolates showed more than 96% removal of nutrients removal from treated wastewater and provided potential scaled-up biomass production of approximately 9.2 to 17.8 tons per hectare per annum. The lipid content of this consortium when cultivated in treated wastewater was approximately 7% wt/wt. About 65% of the algal oil obtained from the consortium could be converted into biodiesel.

Wastewater bioremediation by microalgae provides several advantages as it is an eco-friendly process with no secondary pollution, if the biomass produced is reused; and it allows efficient nutrient recycling (Oswald W. J. (1963) Dev. Ind. Microbiol. 4: 112-119; Olguin E. J. (2003) Biotechnol. Adv. 22: 81-91). Algae are microorganisms capable of performing photosynthesis more efficiently than plants using sunlight and carbon dioxide. The potential biomass productivity of algae under optimum scenario ranges from about 100 to about 150 tons per hectare per annum (Rodolfi et al., (2008) Biotechnol. Bioeng. 102: 100-112; Weyer et al., (2009) Bioenerg. Res. DOI 10.1007/s12155-009-9046-x), a factor 10-15 times higher than the productivity of conventional agricultural crops. Algae do not need soil and can grow in poor quality wastewaters.

Algae have the potential to produce about 40,700-53,200 liters per hectare per annum of oil (Weyer et al., (2009) Bioenerg. Res. DOI 10.1007/s12155-009-9046-x), which is 6 to 8 times better than the yield of oil palm considered currently the best source for the purpose. Oil from algae can be used for biodiesel while residual biomass can be fermented into ethanol and biomethane.

Biofuels derived from plants like algae are considered “carbon neutral”. Two of the most limiting factors to a sustainable and economic production of algae for biofuel purposes are water and fertilizers. Maximum cultivation of algae would require 2 million liters of water per hectare if grown in open ponds, but to compensate for evaporative losses a further 11 million liters would be required. Hence, water management is a critical bottleneck in practical algae cultivation.

Cultivation of algae can also require supplementation of nutrients, particularly nitrogen and phosphorus. Increasing fertilizer costs make economically feasible production of algae a still difficult target. It has been shown that wastewater generated by the carpet industry, when combined with a typical city sewage, can provide a cheap source of an algal culture medium while simultaneously being treated to reduce or remove the industry by-products that are undesired in the environment.

Different cultivation systems such as open raceway ponds and closed photobioreactors (PBR) are currently being used for commercial cultivation of algae. However, such systems have not been considered for the growth of algae on carpet wastewater. In particular, it had been unknown whether the nutritional limitations of a mixed carpet industry wastewater-sewage medium, including any toxic by-products from the carpet manufacturing process itself, would allow the growth of alga as well as yielding algal lipid and biomass that could be used as sources of biofuel (biodiesel). The coloration of the wastewater-sewage culture medium arising especially from the dyes used in carpet manufacture further could have provided an impediment to efficient algal growth by restricting exposure of the cells to sunlight. Accordingly, the embodiments of the present disclosure provide a means of culturing the alga on this type of medium that provides increased irradiance, overcoming the inherent disadvantage of colored wastewater-derived culture medium.

The present disclosure, therefore, provides isolated cultures of algae that show the capacity to survive and proliferate on the wastewater derived from carpet manufacture. In particular, embodiments of the disclosure provide at least two mixed populations of algae that provide growth rates and growth yields that are suitable for the economic production of algal biomass and biodiesel therefrom.

Although the isolated algae and combinations thereof according to the disclosure are able to grow on carpet industry wastewater under a variety of conditions, the embodiments of the disclosure further provide a system for the algal cultivation that overcomes some, at least, of the inherent disadvantages of carpet industry wastewater as a culture medium, and especially the presence of dyes and other colorants that reduce the amount of illumination reaching the algae. It has, therefore, been shown that cultivating the algae in vertically aligned polybags containing carpet industry wastewater and city sewage as a culture medium with a limited diameter maximally exposes the alga to irradiating light, and avoids the evaporative losses that occur with atmospherically open raceways or ponds.

The production of energy in the form of oil (lipids) by algae is more useful than the production of starch. If equal volumes of oil and starch are produced, the oil will contain significantly more energy. For example, the energy content in a typical algal lipid is 9 kcal/gram compared to 4.2 kcal/gram for typical algal starch. In the production of sugars from starch, not all the starch is saccharified into sugars which can be easily fermented, so a portion may be lost as unused sugars. Also, the production of biodiesel from the algal oil is essentially energy-neutral, so nearly all of the energy content of the algal oil is retained in the biodiesel. In contrast, the production of alcohol from biomass or starch is less efficient, especially during the fermentation stage which converts the sugars derived from the biomass or starch into alcohol. Fermentation is exothermic, with heat being generated that must be removed and often wasted. One half of the carbon in the sugar is released during fermentation as carbon dioxide and is therefore not available for fuel energy. For all of these reasons biodiesel production is more efficient overall than bioethanol production, and therefore the goal of highest efficiency and lowest cost is served by maximizing biodiesel production.

Nevertheless, starch-producing or biomass-producing algae are an important aspect of the present disclosure. For example, starch products or sugars converted from algal biomass can be used to produce feed for the oil-producing algae and/or production of ethanol or ethyl acetate for use in transesterification of algal oil. Carbon dioxide released during fermentation can be fed back into the algal growth stage, substantially eliminating at least this form of energy loss in the fermentation process.

Any one or more methods for dewatering an algal biomass can be used including but not limited to, sedimentation, filtration, centrifugation, flocculation, froth floatation, and/or semi-permeable membranes, which can increase the concentration of algae by a factor of about 2, 5, 10, 20, 50, 75, or 100. The dewatering step can be performed serially by one or more different techniques to obtain a concentrated algal composition before extraction of lipids therefrom or before fermentation, pyrolysis and the like for the generation of a biofuel. See, for example, Chapter 10 in Handbook of Microalgal Culture, edited by Amos Richmond, 2004, Blackwell Science, for description of downstream processing techniques. Centrifugation separates algae from the culture media and can be used to concentrate or dewater the algae. Various types of centrifuges known in the art, including but not limited to, tubular bowl, batch disc, nozzle disc, valve disc, open bowl, imperforate basket, and scroll discharge decanter types, can be used. Filtration by rotary vacuum drum or chamber filter can be used for concentrating fairly large microalgae. Flocculation is the collection of algal cells into an aggregate mass by addition of polymers, and is typically induced by a pH change or the use of cationic polymers. Foam fractionation relies on bubbles in the culture media which carries the algae to the surface where foam is formed due to the ionic properties of water, air and matter dissolved or suspended in the culture media. An algal composition of the disclosure can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition. An algal composition can also be described by its moisture level or level of solids, especially when it is in a paste form, such as but not limited to a paste comprising about 1%, 2%, 5%, 10%, 15%, 20%, 25%, 30%, 35%, 40%, 45%, or 50% solids by weight.

Mechanical crushing, for example, an expeller or press, a hexane or butane solvent recovery step, supercritical fluid extraction, or the like can also be useful in extracting the oil from oil vesicles of the oil-producing algae grown using the methods of the disclosure. Alternatively, mechanical approaches can be used in combination with biological agents in order to improve reaction rates and/or separation of materials.

Once the oil has been released from the algae it can be recovered or separated from a slurry of algae debris material, e.g. cellular residue, oil, enzyme, by-products, etc. This can be done by sedimentation or centrifugation, with centrifugation generally being faster. Starch production can follow similar separation processes. Recovered algal oil can be collected and directed to a conversion process. The algal biomass left after the oil is separated may be fed into the depolymerization stage described below to recover any residual energy by conversion to sugars, and the remaining husks can be either burned for process heat or sold as an animal food supplement or fish food.

Algal oil can be converted to biodiesel through a process of direct hydrogenation or transesterification of the algal oil. Algal oil is in a similar form as most vegetable oils, which are in the form of triglycerides. This form of oil can be burned directly. However, the properties of the oil in this form are not ideal for use in a diesel engine, and without modification, the engine will soon run poorly or fail. In accordance with the present disclosure, the triglyceride is converted into biodiesel, which is similar to but superior to petroleum diesel fuel in many respects.

One process for converting the triglyceride to biodiesel is transesterification, and includes reacting the triglyceride with alcohol or other acyl acceptor to produce free fatty acid esters and glycerol. The free fatty acids are in the form of fatty acid alkyl esters. Transesterification can be done in several ways, including biologically and/or chemically. The biological process uses an enzyme known as a lipase to catalyze the transesterification, while the chemical process may use, but is not limited to, a synthetic catalyst that may be either an acid or a base. With the chemical process, additional steps are needed to separate the catalyst and clean the fatty acids. In addition, if ethanol is used as the acyl acceptor, it must be essentially dry to prevent production of soap via saponification in the process, and the glycerol must be purified. Either or both of the biological and chemically-catalyzed approaches can be useful in connection with the processes of the present disclosure.

Algal triglyceride can also be converted to biodiesel by direct hydrogenation. In this process, the products are alkane chains, propane, and water. The glycerol backbone is hydrogenated to propane, so there is substantially no glycerol produced as a byproduct. Furthermore, no alcohol or transesterification catalysts are needed. All of the biomass can be used as feed for the oil-producing algae with none needed for fermentation to produce alcohol for transesterification. The resulting alkanes are pure hydrocarbons, with no oxygen, so the biodiesel produced in this way has a slightly higher energy content than the alkyl esters, degrades more slowly, does not attract water, and has other desirable chemical properties.

Accordingly, one aspect of the present disclosure encompasses methods of generating an algal biomass, comprising: (a) forming an algal culture by combining: (i) a population of algal cells characterized as proliferating in a medium comprising carpet industry wastewater, and (ii) a culture medium comprising carpet industry wastewater and a sewage system effluent; and (b) maintaining the algal culture under conditions suitable for the proliferation of the population of algal cells, thereby forming an algal biomass.

In embodiments of this aspect of the disclosure, the medium, before receiving the population of algal cells is treated in a wastewater treatment plant.

In the embodiments of the methods of this aspect of the disclosure, the population of algal cells can comprise at least one of the group consisting of: a marine algal strain, a freshwater (non-marine) algal strain, a cyanobacter strain, a diatomaceous algal strain, a plurality of marine algal strains, a plurality of freshwater (non-marine) algal strains, a plurality of cyanobacter strains, and a plurality of diatomaceous algal strains, or any combination thereof.

In the embodiments of this aspect of the disclosure, at least one algal strain of the population of algal cells can be isolated from a source in contact with the wastewater effluent of the carpet industry.

In the embodiments of this aspect of the disclosure the population of algal cells can comprise an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

In the embodiments of this aspect of the disclosure, at least one algal strain of the population of algal cells can be selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In some embodiments of this aspect of the disclosure, the population of algal cells comprises at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

In some embodiments, the population of algal cells can comprise a plurality of strains selected from the Group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

In an embodiment of this aspect of the disclosure, the population of algal cells can be a consortium, where the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, Chlamydomonas spp. In an embodiment of this aspect of the disclosure, the population of algal cells can be a consortium comprising Chlamydomonas globosa, Chlorella minutissima, and Scenedesmus bijuga.

In the embodiments of this aspect of the disclosure, the algal culture can be contained within a raceway, a vertical tower reactor, or a polybag, and wherein the algal culture can be optionally provided with air supplemented with carbon dioxide.

In the embodiments of this aspect of the disclosure, the method can further comprise isolating the algal biomass from the medium.

Another aspect of the disclosure encompasses methods of producing a biofuel from carpet industry wastewater comprising: (a) forming an algal culture by combining: (i) a population of algal cells characterized as proliferating in a medium comprising carpet industry wastewater, and (ii) a culture medium comprising carpet industry wastewater and a sewage system effluent; (b) maintaining the algal culture under conditions suitable for proliferation of the population of algal cells, thereby forming an algal biomass; (c) isolating the algal biomass from the medium; and (d) obtaining from the isolated algal biomass a biofuel or a source of a biofuel, wherein the step of obtaining from the isolated biomass a biofuel comprises the steps of isolating a lipid material from the biomass or converting the biomass to a biofuel, and wherein the isolated lipid material may be converted to a biofuel.

In embodiments of this aspect of the disclosure, the medium, before receiving the population of algal cells is treated in a wastewater treatment plant.

In embodiments of this aspect of the disclosure, the population of algal cells can comprise at least one of the group consisting of: a marine algal strain, a freshwater (non-marine) algal strain, a cyanobacter strain, a diatomaceous algal strain, a plurality of marine algal strains, a plurality of freshwater (non-marine) algal strains, a plurality of cyanobacter strains, a plurality of diatomaceous algal strains, or any combination thereof.

In embodiments of this aspect of the disclosure, at least one algal strain of the population of algal cells is isolated from a source in contact with the wastewater effluent of the carpet industry.

In embodiments of this aspect of the disclosure, the population of algal cells can comprise an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

In other embodiments of this aspect of the disclosure, at least one algal strain of the population of algal cells can be selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In yet other embodiments of this aspect of the disclosure, the population of algal cells can comprise at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

In still other embodiments of this aspect of the disclosure, the population of algal cells can comprise a plurality of strains selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

In one embodiment of the disclosure, the population of algal cells can be a consortium, where the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, Chlamydomonas spp. In an embodiment of this aspect of the disclosure, the population of algal cells is a consortium comprising Chlamydomonas globosa, Chlorella minutissima, and Scenedesmus bijuga.

In another embodiment of this aspect of the disclosure, the population of algal cells comprises Chlamydomonas globosa, Chlorella minutissima, and Scenedesmus bijuga.

In embodiments of this aspect of the disclosure, the algal culture can be contained within a raceway, a vertical tower reactor, or a polybag, and wherein the algal culture is optionally provided with air supplemented with carbon dioxide.

Still another aspect of the disclosure encompasses a system for generating an algal biomass, the system comprising an algal culture container selected from a raceway, a vertical tower reactor, a polybag, or a plurality of any thereof, and where the container or plurality of containers is optionally provided with an air supply supplemented with carbon dioxide; an algal culture medium comprising carpet industry wastewater and optionally a sewage system effluent; and a population of algal cells in the algal culture medium, where the algal cells can be selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In embodiments of this aspect of the disclosure, the system can comprise a plurality of polybags.

In embodiments of this aspect of the disclosure, the population of algal cells can be a consortium, where the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, Chlamydomonas spp. In an embodiment of this aspect of the disclosure, the population of algal cells comprises Chlamydomonas globosa, Chlorella minutissima, and Scenedesmus bijuga.

Yet another aspect of the disclosure comprises embodiments of an isolated population of algal cells comprising at least one algal strain isolated from a source in contact with the wastewater effluent of the carpet industry and capable of proliferating on a medium comprising carpet industry wastewater.

In embodiments of this aspect of the disclosure, at least one algal strain of the population of algal cells can be selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, a Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In some embodiments, the population of algal cells can comprise an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

In some embodiments of this aspect of the disclosure, the algal population can comprise at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

In some embodiments of this aspect of the disclosure, the algal population can comprise a plurality of strains selected from the Group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

In one embodiments of the disclosure, the population of algal cells can be a consortium, where the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, Chlamydomonas spp. In an embodiment of this aspect of the disclosure, the population of algal cells comprises Chlamydomonas globosa, Chlorella minutissima, and Scenedesmus bijuga.

In other embodiments of the disclosure, the population of algal cells can be a consortium comprising Chlamydomonas globosa, Chlorella minutissima, and Scenedesmus bijuga.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Wastewater Collection and Analysis

Collection: The wastewaters used in the study were collected from a local utility company in North Georgia, U.S.A. treating 110-150 million liters of wastewater per day and which contained about 85-90% industrial wastewater, mainly from carpet and rug mills and the rest being typical sanitary sewage water from the area. Process chemicals used in the carpet mills and sewage contributed to the organic and inorganic load of the wastewaters. Due to the variation of wastewater composition with collection time, the wastewater samples were collected in a large batch of 1000 L capacity totes and samples required for the experiments were stored in 20 L buckets in a cold room maintained at 4° C. For characterization of treated and untreated wastewater, samples were periodically collected from the treatment facility in all seasons.
Wastewater characterization: Treated and untreated wastewaters received from a local utility company in Georgia, U.S.A. were periodically analyzed for physico-chemical characteristics to monitor the change in nutrient concentration throughout the year. Biochemical Oxygen Demand (BOD), Chemical Oxygen Demand (COD), Total Suspended Solids (TSS), Total Dissolved Solids (TDS), Total Volatile Solids (TVS), Total Solids (TS) and Total Kjeldahl Nitrogen (TKN) showed a reduction in the treated wastewater, as shown in Table 1.

TABLE 1
Characteristics of wastewater obtained from different carpet mills, combined
carpet industry untreated wastewater and standard algal growth medium

| | NO₃—N | NH₄—N | PO₄—P | Total N | Total P |
|---|---|---|---|---|---|
| Type of wastewater | (mg L⁻¹) | | | | |
| Carpet industry | 0.009-0.327 | 0.020-45.668 | 0.003-10.69 | na | na |
| wastewater (untreated) ^{a} | | | | | |
| Carpet industry | 17.58-25.85 | 0.21-28.13 | 5.3 | 32.6-45.9 | 5.47-13.83 |
| wastewater (treated) ^{b} | | | | | |
| Standard algal growth | 263 | 0.3 | 5.9 | 270 | 5.9 |
| medium (BG11) | | | | | |

na—not analyzed;
^{a} Collected from the outlet of 12 carpet mills;
^{b} Contained 10-15% city sewage mix

Amounts of phosphorus appeared sufficient to support algal growth in both untreated as well as treated wastewaters. Both untreated and treated wastewater as used herein had an N:P ratio of between about 4.06:1 and about 0.83:1, which indicated a N limitation (Table 1). Total nitrogen was less in treated wastewater but appeared sufficient (32.6-45.9 mg L⁻¹) to support the growth of microalgae in untreated wastewater. Other parameters did not have levels high enough to be toxic to native algae.

Preparation of wastewater: Upon receipt of the carpet industry wastewater, approximately 175 mL of bleach containing 6.15% sodium hypochlorite was added to 1000 L of carpet industry untreated wastewater for sterilization. The wastewater totes were kept under tarps and out of direct sunlight. For each round of the study the wastewater was filtered twice, first by pumping from the container through a WaterCo Commandomatic bag filter housing fitted with 50 μm mesh filter into a separate 1200 L storage tote.

To achieve a visible clarity it was pumped for approximately 1 h through a Hayward Perflex diatomaceous earth filter containing Celatom diatomaceous earth media. Immediately before inoculation, residual chlorine concentration in the wastewater was determined with a Lamotte Smart2 colorimeter using N,N Diethyl-1,4 Phenylenediamine Sulfate (DPD) method and pre-packaged unit dose vials (APHA-AWA-WEF, 2005).

### Example 2

### Algal Culturing Systems

Bioreactors: Raceway ponds were made of opaque plastic and were 1.52 m wide, 2.44 m long and 0.61 m deep with a capacity of about 2000 L. The working volume maintained in the raceway ponds was 950 L, 550 L, and 500 L, in runs 1 & 2, 3 and 4, respectively, of Table 2.

TABLE 2
Experiments conducted in greenhouse using carpet industry
untreated wastewater growth medium in raceway ponds (RW),
vertical tank reactors (VT) and polybags (PB)

| | | | | Total | | |
|---|---|---|---|---|---|---|
| | | Inoculum | | volume | Depth | Air flow |
| Run | Reactor | Algae | Ratio (L) | (L) | ^{a} (cm) | (L min⁻¹) |
| 1 | RW | Cg/Cm/Sb | 50/50/50 | 950 | 30 | 10 |
| 2 | RW | Cg/Cm/Sb | 50/50/50 | 950 | 20 | 10 |
| 3 | RW | Cg/Cm/Sb | 18/18/18 | 550 | 18 | 10 |
| | VT | | 4/4/4 | 100 | 61 | 1.8 |
| 4 | RW | Cg/Cm | 25/25/25 | 500 | 15 | 10 |
| | VT | | 5/5 | 100 | 61 | 2 |
| | PB | | 1/1 | 20 | 95 | 0.4-0.8 |

Cg—Chlamydomonas globosa; Cm—Chlorella minutissima; Sb—Scenedesmus bijuga;
^{a} Depth of water column in each reactor

In each raceway pond there was a paddle wheel that operated at 20-30 cm depth to generate a flow rate of approximately 21±3 cm s⁻¹. Vertical tank reactors (VTRs) were 0.45 m in diameter, 1.52 m height, had a 100 L working volume, and made of transparent acrylic sheets.

In a third system, a roll of low density polyethylene (LDPE-Uline-6-Mil heavy duty polytubing with 50.8 cm circumference) material was used to fabricate hanging polybags (95 cm deep, 15 cm diameter, 20 L working volume). All the reactor types were supplied with delivery tubings and air stones (spargers) for bubbling the 5-6% CO₂-air mixture through the medium. CO₂ supply: Supplemental CO₂ was blended with atmospheric air using a Concoa BlendMaster Model 1000 mixer and passed through a Whatman HEPA-Vent filter at about 5-6% CO₂ concentration in air. Rotameters were used to regulate air flow rates among the raceways, VTRs and polybags. For the VTRs, mixing was accomplished by bubbling CO₂ and air mixture through rectangular air stones (15×4×4 cm), whereas for the polybags, the mixture was bubbled into a port disk (0.72 cm opening) sealed into place at the bag's bottom. To keep the VTRs and polybags stirred after terminating the supply of supplemental CO₂, each evening ambient air was pumped into these cultures at the same flow rate as that of the supplemental CO₂ gas mix during the day. Culture temperature and pH for the raceways, VTRs and polybags were determined daily.

The VTRs were arranged in a row parallel in an East-West direction. Polybags were arranged East to West while two were positioned North to South. Two bags hung 0.20 m apart and the other two were at 0.55 m distance.

Initiating cultures: Wastewater was pumped from the storage tote through a diatomaceous earth filter into raceways and VTRs. Algal inoculum was maintained in VTRs in BG11 medium for the purpose of inoculation. Inoculum was drained from the bottom of vertical tank reactors through a gate valve fitted with a 1.3 cm internal diameter garden hose that was sent either directly into the raceways or into pre-autoclaved Glass carboys/Erlenmeyer flasks for subsequent delivery to the intended culture system.
Harvesting: Biomass was harvested at 2250×g and dried at 40° C. in a hot air oven for 72 h. It was subsequently stored at 4-5° C.

### Example 3

Microalgae identification, diversity and community composition: Original wastewater samples collected during different seasons were immediately preserved at 4° C. after the addition of 25% aqueous general grade glutaraldehyde (1 mL per 100 mL of wastewater). Identification of algal taxa and biovolume assay according to Charles et al., (2002) (Report No. 02-06, Patrick Center for Environmental Research, The Academy of Natural Sciences, Philadelphia, Pa. pp 124, incorporated herein by reference in its entirety).
Isolation of microalgae: Untreated and treated wastewater samples and soil samples collected from the wastewater land application sites were used as the sources for isolating native algal strains. BG11 was used as the enrichment and isolation medium (Stanier et al., (1971) Bacteriol. Rev. 35: 171-205).

For enrichment experiments, 50 mL of BG11 medium (nitrogen-free and supplemented with sodium nitrate) were dispensed into 250 mL Erlenmeyer flasks and sterilized. Soil (5 g) from the land application site of the local utility company's treated water and homogenized wastewater samples (5 mL each of treated and untreated wastewater) were mixed separately with the medium and agitated for 30 min on a rotary shaker. The flasks were then incubated for enrichment at 25±1° C. under a light intensity of 75-80 μmol photon m⁻² s⁻¹ and L:D cycles of 12:12 h for 3 weeks. Algae were isolated by serial dilution technique. One mL of the culture from tubes showing algal growth in highest dilution tubes was spread-plated on BG11 agar plates. The plates were incubated for 2 weeks and after the colony formation, isolated single colonies were picked up and maintained on the BG11 agar slants.

### Example 4

Diversity and community composition of microalgae in carpet industry wastewater: The composition of algal communities was assessed in carpet industry wastewaters (treated and untreated) for all four seasons, as shown in Table 3.

TABLE 3
Seasonal variations in the microalgal diversity and community composition in
treated (T) and untreated (U) carpet industry wastewaters (in % biovolume).

| | Summer | | Fall | | Winter | | Spring | |
|---|---|---|---|---|---|---|---|---|
| Genus | T | U | T | U | T | U | T | U |
| Chlorophyta | | | | | | | | |
| Chlamydomonas sp. | — | — | 0.89 | 88.83 | 1.57 | — | — | — |
| Chlorella vulgaris | — | — | 4.11 | — | 0.35 | — | — | — |
| Chlorococcaceae sp. | — | — | — | — | — | — | 12.79 | 0.72 |
| Chlorococcum humicola | 0.11 | 77.59 | 4.23 | 5.5 | — | 83.95 | — | — |
| Coelastrum microporum | — | — | 1.3 | — | — | — | 5.62 | — |
| Gloeocystis vesiculosa | 1.46 | — | 5.19 | — | — | — | — | — |
| Monoraphidium mirabile | — | — | 7.97 | — | — | — | — | — |
| Oedogonium sp. | — | — | — | — | — | — | 0.23 | — |
| Oocystis lacustris | — | — | 7.75 | 1.21 | — | — | — | — |
| Scenedesmus abundans | — | — | — | — | — | — | 0.09 | — |
| Scenedesmus acuminatus | — | — | — | — | — | — | 2.61 | — |
| Scenedesmus acutus | — | — | — | — | — | — | 3.42 | 11.84 |
| Scenedesmus acutus alternans | — | — | — | — | — | — | 0.75 | — |
| Scenedesmus bicaudatus | — | — | — | — | — | — | 0.16 | — |
| Scenedesmus bijuga | — | — | 2.11 | 0.42 | — | — | 1.59 | — |
| Scenedesmus bijuga alternans | — | — | — | — | — | — | 0.23 | — |
| Scenedesmus denticulatus | — | — | — | — | — | — | 0.13 | — |
| Scenedesmus dimorphus | — | — | — | — | 92.97 | 1.61 | 1.87 | — |
| Scenedesmus incrassatulus | — | — | — | — | 1.3 | — | — | — |
| Scenedesmus obliquus | — | — | — | — | — | — | 0.67 | 0.06 |
| Scenedesmus quadricauda | — | — | 2.17 | | 1.48 | — | 1.29 | — |
| Scenedesmus quadrispina | — | — | — | — | — | — | 3.54 | — |
| Scenedesmus serratus | — | — | — | — | — | — | 0.09 | — |
| Stigeoclonium sp. | — | — | — | — | — | — | — | 38.84 |
| Ulothrix variabilis | 0.03 | — | — | — | — | — | — | — |
| Uroglena sp. | — | — | — | — | — | — | — | 46.49 |
| Chlorophyta contribution | 1.6 | 77.59 | 35.72 | 95.96 | 97.67 | 85.56 | 35.08 | 97.95 |
| Cyanophyta | | | | | | | | |
| Anabaena sp | 54.09 | — | — | — | — | — | — | — |
| Aphanocapsa delicatissima | — | — | — | — | — | — | — | 0.02 |
| Aphanocapsa hyalina | 0.02 | — | — | — | — | — | — | — |
| Aphanothece sp. | — | — | — | — | — | — | — | 0.12 |
| Calothrix braunii | 2.06 | — | — | — | — | — | — | — |
| Chroococcaceae sp. | — | — | — | — | — | — | 0.02 | 0.81 |
| Chroococcus minutus | — | — | — | — | — | — | 0.05 | — |
| Cylindrospermopsis sp. | 4.65 | — | — | — | — | — | — | — |
| Leibleinia kryloviana | — | — | — | 0.47 | — | 7.93 | — | — |
| Limnothrix sp. | — | 0.04 | — | — | — | — | — | |
| Limnothrix redekei | — | — | — | 2.8 | 1.03 | 6.51 | — | — |
| Lyngbya sp. | — | — | — | — | — | — | 36.74 | — |
| Nostoc sp. | 5.86 | — | — | — | — | — | — | — |
| Oscillatoria sp. | 20.34 | — | — | — | — | — | — | — |
| Oscillatoria tenuis | — | 22.37 | — | — | — | — | 1.59 | — |
| Planktolyngbya limnetica | 11.09 | — | — | — | — | — | — | — |
| Raphidiopsis curvata | — | — | 64.28 | 0.76 | — | — | — | — |
| Synechococcus elongatus | — | — | — | — | — | — | — | 0.11 |
| Synechococcus sp. | — | — | — | — | — | — | 0.06 | 0.35 |
| Synechocystis sp. | — | — | — | — | — | — | — | 0.17 |
| Cyanophyta contribution | 98.11 | 22.41 | 64.28 | 4.04 | 1.03 | 14.44 | 38.46 | 1.72 |
| Bacillariophyta | | | | | | | | |
| Eunotia sp. | — | — | — | — | — | — | 1.49 | — |
| Navicula pelliculosa | — | — | — | — | — | — | 3.12 | — |
| Navicula sp. | — | — | — | — | 1.3 | — | — | — |
| Nitzschia palea | — | — | — | — | — | — | 21.85 | 0.33 |
| Nitzschia amphibia | 0.09 | — | — | — | — | — | — | — |
| Nitzschia pura | 0.02 | — | — | — | — | — | — | — |
| Gomphonema parvulum | 0.02 | — | — | — | — | — | — | — |
| Gomphonema gracile | 0.01 | — | — | — | — | — | — | — |
| Bacillariophyta contribution | 0.14 | 0 | 0 | 0 | 1.3 | 0 | 26.46 | 0.33 |
| Cryptophyta | | | | | | | | |
| Rhodomonas sp. | 0.15 | — | — | — | — | — | — | — |
| Cryptophyta contribution | 0.15 | 0 | 0 | 0 | 0 | 0 | 0 | 0 |

Twenty-seven species of green algae, 20 species of cyanobacteria, and 8 species of diatoms were observed in both treated and untreated wastewaters. In terms of biovolume, green algae (chlorophyta) and cyanobacteria (cyanophyta) were the two major groups of algae dominating both untreated and treated wastewaters in all seasons followed by diatoms (Bacillariophyta). Green algae dominated untreated wastewater in all seasons and treated wastewater during winters, whereas cyanobacteria dominated treated wastewater in summer and fall (Table 3). The genus Scenedesmus had the highest species richness, being represented by 14 species (Table 3). As in natural freshwater systems, there was a tendency for the seasonal fluctuation in algal flora of the wastewaters. It has also been established that toxic chemical stress causes large changes in community structure (Howarth R. W. (1991) in: Cole et al., (Eds.), Comparative Analysis of Ecosystems: Patterns, Mechanisms and Theories. Springer Verlag, New York Inc., pp. 169-196).

### Example 5

Microalgal strain isolation and development of consortium: Fifteen isolates were obtained from the carpet industry wastewaters and soil and analysed for their lipid content. The isolates were mostly green algal species such as Chlorella, Chlamydomonas, Scenedesmus, and Gloeocystis and cyanobacterial species such as Anabaena and Limnothrix. Maximum lipid (16 and 13%) content was found in two strains of Gloeocystis, whereas the other species all contained less than 9% lipids.

Fifteen isolates were obtained from the wastewaters and analysed for their neutral lipid content (Table 4). Two strains of Gloeocystis vesiculosa showed maximal (approximately 16% and 13%, respectively) lipid content. The rest all contained less than 9% lipids. All these strain were isolated from treated and raw (untreated wastewater) and soil and were grown in pure cultures or mixed together in equal quantities at an OD value of 0.7 to form a primary consortium of mixed strains of algae.

TABLE 4
Strains isolated from raw and treated wastewaters and soil from
land application sites and their lipid contents.

| Isolate No. | Strain | Lipid (%) |
|---|---|---|
| 1 | Gleocytis vesiculosa strain 1 | 6.62 |
| 2 | Limnothrix redekei | 5.52 |
| 3 | Gleocytis vesiculosa strain 2 | 15.59 |
| 4 | Scenedesmus spp. | 6.8 |
| 5 | Limnothrix redekei | 5.09 |
| 6 | Chlorococcum humicola strain 1 | 8.07 |
| 7 | Chlorococcum humicola strain 2 | 2.88 |
| 8 | Chlorococcum humicola strain 3 | 3.49 |
| 9 | Clorella vulgaris strain 1 | 1.59 |
| 10 | Clorella vulgaris strain 2 | 3.97 |
| 11 | Clorella vulgaris strain 3 | 2.60 |
| 12 | Gleocytis vesiculosa strain 3 | 12.73 |
| 13 | Anabaena spp. | 7.97 |
| 14 | Gleocytis vesiculosa strain 4 | 7.14 |
| 15 | Chlamydomonas spp. | 3.53 |

Three algal strains: Chlamydomonas globosa, Chlorella minutissima, and Scenedesmus bijuga, all isolated from carpet industry wastewater, were maintained as a consortium in BG11 medium by frequent subculturing in a growth room at 25±2° C. under approximately 80 μmol of photons m⁻² s⁻¹ light intensity with a 12:12 h L/D cycle.

### Example 6

Preliminary screening: Thirteen microalgal strains (Table 5), and the preliminary consortium of wastewater isolates (from Table 4), were screened for their growth responses in terms of their chlorophyll a content.

TABLE 5
Strains used in the preliminary screening (all strains designated as
UTEX are available from the Austin University Culture Collection)

| | | Standard |
|---|---|---|
| | | Growth |
| Strain | Form | Medium |
| Botryococcus braunii UTEX 572 | Fresh | BG11 |
| Chlorella protothecoides UTEX 25 | Fresh | BG11 |
| Chlorella saccharophila var. saccharophila | Fresh | BG11 |
| UTEX 2469 | | |
| Chlorella vulgaris UTEX 2714 | Fresh | BG11 |
| Cricosphaera carterae UTEX LB1014 | Marine | Modified BG11 |
| Dunaliella tertiolecta UTEX LB999 | Marine | Modified BG11 |
| Nannochloris oculata UTEX LB1998 | Marine | Modified BG11 |
| Spirulina platensis UTEX LB1926 | Marine | Modified BG11 |
| Spirulina maxima UTEX LB2342 | Fresh | Modified |
| Tetraselmis suecica UTEX LB2286 | Marine | Modified BG11 |
| Tetraselmis chuii UTEX LB232 | Marine | Modified BG11 |
| Phaeodactylum tricornutum UTEX 646 | Marine | Modified BG11 |
| Pleurochrysis carterae CCMP 647 | Marine | Modified BG11 |
| Preliminary Consortium of wastewater | Fresh | BG11 |
| isolates (from Table 4) | water | |

BG 11 and modified CFTRI medium (Venkataraman et al., (1982) Phykos 21: 56-62) were used to cultivate fresh water strains whereas marine algal strains were maintained in modified BG11 medium prepared in filtered sea water and supplemented with 0.5 mL L⁻¹ of vitamin mix (cyanocobalamin, 0.001 g L⁻¹; thiamine HCl, 2 g L⁻¹; biotin, 0.001 g L⁻¹).

The consortium of native algal isolates was prepared by mixing equal quantities of 13 wastewater isolates with a biomass concentration of approximately 0.1 g L⁻¹ each. Preliminary experiments were conducted in test tubes containing 15 mL of filtered and sterilized treated and untreated wastewater as growth medium with standard algal growth medium as control.

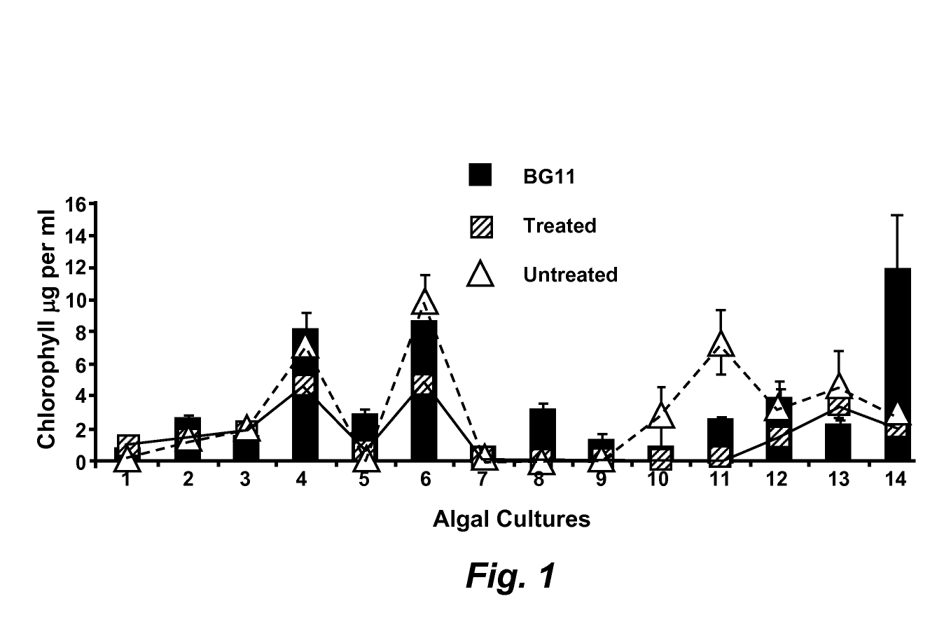

Growth after 10 days was estimated in terms of chlorophyll a content. Among all the algal strains tested, P. carterae (3.4 μg mL⁻¹), B. braunii (0.9 μg mL⁻¹) and C. saccharophila (1.8 μg mL⁻¹) recorded 56%, 26% and 23% increases, respectively. In chlorophyll a content in treated wastewater, respectively over the standard BG11 medium, T. suecica (2.8 μg mL⁻¹), T. chuii (7.3 μg P. carterae (4.7 μg mL⁻¹), C. Saccharophila (2.0 μg mL⁻¹), and D. tertiolecta (9.9 μg mL⁻¹) recorded 247%, 190%, 118%, 36%, and 16% increases in chlorophyll a, respectively in untreated wastewater over the control, as shown in FIG. 1. The preliminary consortium of native isolates from wastewaters recorded the highest chlorophyll a content of 11.9 μg mL⁻¹ in the standard medium when compared to all other algal cultures and treatments (FIG. 1). The preliminary consortium recorded chlorophyll a content of 2.1 μg mL⁻¹ and 2.9 μg mL⁻¹ in treated and untreated wastewaters, respectively.

Based on the growth responses of the strains, three fresh water algal cultures (B. braunii, C. saccharophila, and the preliminary consortium) along with two marine algal cultures (D. tertiolecta and P. carterae) were studied further. Even though T. suecica and T. chuii showed good growth in untreated wastewater, the present study evaluated treated carpet industry wastewater for biodiesel production.

Both treated and untreated carpet industry wastewaters supported the growth of certain marine algal forms without any salt supplementation. The present data, therefore, show that certain the marine algal strains can grow in carpet industrial wastewaters, suggesting unique osmotic adjustment and regulation mechanisms to tolerate the hypo-osmotic stress conditions. The results of this study show that selected high-lipid marine algal strains can be cultivated on industrial, and municipal and agricultural wastewater for biofuel applications.

### Example 7

Biomass production and nutrient removal potential of the consortium: An experiment aimed at examining biomass production and nutrient removal potential of the consortium was carried out under 2 different levels of CO₂ (ambient and 6%) and temperature (15° C. and 25° C.). Filtered (50 μm mesh) and sterilized treated wastewater was used as nutrient medium with an initial pH of 7. The experiment was conducted in 1 L capacity Erlenmeyer flasks with 500 mL growth medium in triplicates. The consortium was prepared as described in Example 6 and inoculated to achieve an initial concentration of approximately 0.1 g L⁻¹. The flasks were incubated in a temperature-controlled water bath under continuous fluorescent illumination at an irradiance of 75-80 μmol photons m⁻² s⁻¹. Filtered (1-μm filter) ambient air and a 6% CO₂-air mixture were bubbled through the growth medium at a rate of 100 mL min⁻¹.

B. braunii, C. saccharophila, D. tertiolecta, P. carterae and the consortium were selected for a time-scale study to evaluate their biomass and lipid production potential in carpet industry wastewaters (treated and untreated wastewater) in comparison with standard growth medium, as shown in Table 6.

TABLE 6
Biomass and oil production potential of B. braunii, C. saccharophila,
D. tertiolecta, P. carterae, and a consortium of algal isolates
in treated and untreated carpet industry wastewaters

| | | | | Estimated | |
|---|---|---|---|---|---|
| | | | | biomass | Estimated oil |
| | | Biomass | Lipids | productivity | yield |
| Culture | Medium | (g L⁻¹ d⁻¹) | (%) | (t ha⁻¹ year⁻¹) | (L ha⁻¹ year⁻¹⁾ |
| Botryococcus | BG11 | 0.019 ± 0.003 | 13.50 ± 3.78 | 13.7 | 2109 |
| braunii | Treated | 0.037 ± 0.005 | 9.50 ± 1.24 | 26.3 | 2839 |
| | Untreated | 0.034 ± 0.007 | 13.20 ± 1.85 | 24.5 | 3675 |
| Chlorella | BG11 | 0.018 ± 0.004 | 12.90 ± 1.16 | 12.7 | 1869 |
| saccharophila | Treated | 0.016 ± 0.003 | 17.00 ± 2.89 | 11.4 | 2194 |
| | Untreated | 0.023 ± 0.004 | 18.10 ± 1.27 | 16.1 | 3319 |
| Dunaliella | Modified | 0.031 ± 0.008 | 12.80 ± 0.64 | 22.1 | 3216 |
| tertiolecta | BG11 | | | | |
| | Treated | 0.038 ± 0.003 | 12.20 ± 1.41 | 26.9 | 3728 |
| | Untreated | 0.028 ± 0.005 | 15.20 ± 2.43 | 20.3 | 3510 |
| Pleurochrysis | Modified | 0.028 ± 0.004 | 9.70 ± 1.26 | 20.3 | 2240 |
| carterae | BG11 | | | | |
| | Treated | 0.037 ± 0.006 | 11.80 ± 2.10 | 26.3 | 3526 |
| | Untreated | 0.033 ± 0.005 | 12.00 ± 0.80 | 23.9 | 3260 |
| Preliminary | BG11 | 0.027 ± 0.007 | 10.90 ± 2.62 | 19.1 | 2369 |
| Consortium | Treated | 0.041 ± 0.005 | 12.20 ± 1.33 | 29.3 | 4060 |
| | Untreated | 0.039 ± 0.009 | 12.00 ± 2.12 | 28.1 | 3830 |

Except for Chlorella saccharophila, all the strains had higher yields in treated wastewater than in standard growth medium. In untreated wastewater however, Dunaliella tertiolecta did not perform better than on modified BG 11 medium (Table 6). In comparison to all unialgal cultures, the consortium performed the best in treated wastewater. It was the most potent candidate with the potential to generate 29.3 tons of biomass and approximately 4,060 L of oil ha⁻¹ year⁻¹ (Table 6).

In untreated wastewater the preliminary consortium has the potential to produce approximately 28.1 tons of biomass and approximately 3,830 L of oil ha⁻¹ year⁻¹ (Table 6). The marine algal forms Dunaliella tertiolecta and Pleurochrysis carterae were estimated to produce 26.9 and 26.3 tons of biomass ha⁻¹ year⁻¹ in treated wastewater, respectively (Table 6). Biomass and oil productivity were estimated based on the volumetric biomass and lipid production in batch and static culture experiments conducted in 250 ml Erlenmeyer flasks incubated under 75-80 μmol m⁻² s⁻¹ of light intensity with no CO₂ supplementation.

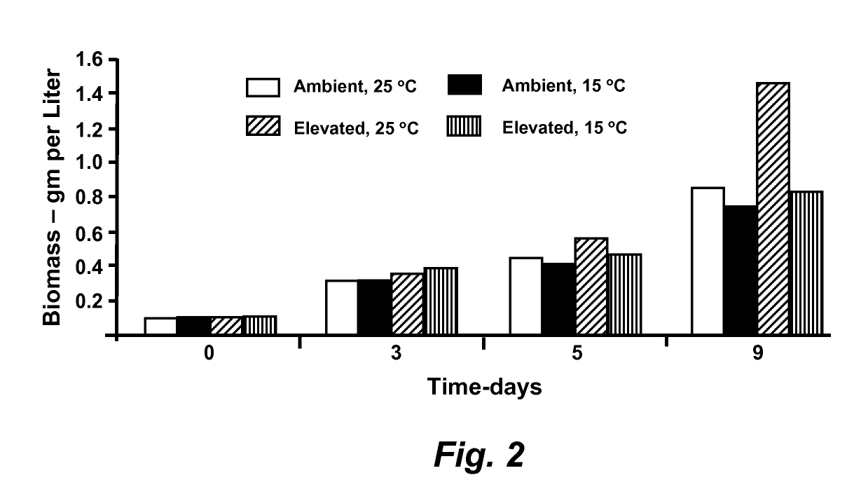

CO₂ bubbling at 25° C. showed significant increases after 3 days of inoculation and on ninth day, the productivity was 1.47 g L⁻¹, a 12.5 fold increase over the initial level of biomass (FIG. 2) and 1.8 fold higher than that with an ambient level of CO₂ and 25° C. Biomass productivity of the preliminary consortium grown at elevated CO₂ and 15° C. was similar to the biomass productivity at ambient level of CO₂ and 25° C., as shown in FIG. 2. The growth of preliminary consortium at ambient air and 15° C. recorded lowest biomass productivity among all treatments. Accordingly, the data supports that the preliminary consortium was robust and could tolerate even low temperature conditions.

The preliminary consortium growth was also significant in treated wastewater despite its low N and P concentrations. Nitrate-N, ammonia-N, and phosphate-P in the culture medium were depleted by about 99%, 100%, and 75%, respectively in the first 24 h of incubation under all conditions of treatment, as shown in Table 7.

TABLE 7
Nutrient removal potential of consortium of native algal isolates in treated wastewater.

| | | | | | | | Removal | Removal |
|---|---|---|---|---|---|---|---|---|
| | Days | | | | | | after 24 | after 72 |
| Treatments | 0 | 1 | 3 | 5 | 7 | 9 | h (%) | h (%) |

Nitrate-N removal (mg L⁻¹)

| | | | | | | | | |
|---|---|---|---|---|---|---|---|---|
| T1 | 2.832 | na | 0.0097 | 0.0041 | 0.0035 | 0.0032 | na | 99.7 |
| T2 | 2.832 | na | 0.0045 | 0.0039 | 0.0034 | 0.0035 | na | 99.8 |
| T3 | 2.832 | 0.0073 | 0.0051 | 0.0048 | 0.0046 | 0.0043 | 99.7 | 99.8 |
| T4 | 2.832 | 0.006 | 0.0045 | 0.0043 | 0.0036 | 0.0034 | 99.8 | 99.8 |

Phosphate-P removal (mg L⁻¹)

| | | | | | | | | |
|---|---|---|---|---|---|---|---|---|
| T1 | 4.807 | na | 0.0414 | 0.0509 | 0.0253 | 0.0149 | na | 99.1 |
| T2 | 4.807 | na | 0.0576 | 0.0441 | 0.0345 | 0.0201 | na | 98.8 |
| T3 | 4.807 | 1.1843 | 0.1654 | 0.0344 | 0.0213 | 0.0143 | 75.4 | 96.6 |
| T4 | 4.807 | 1.128 | 0.1615 | 0.0337 | 0.019 | 0.0153 | 76.5 | 96.6 |

na—not analyzed.

T1 and T2 were the treatments bubbled with ambient air and incubated at 25° C. and 15° C., respectively. T3 and T4 were the treatments bubbled with 6% CO₂ enriched air and incubated at 25° C. and 15° C., respectively. Ammonia-N that was 0.761 mg L⁻¹ in treated wastewater on day 0 was brought to nil the next day in all four treatments.

By 72 h, nitrate-N removal was 99.7-99.8% and phosphate-P removal reached 98.8-99.1% at ambient air and 96.5% under elevated CO₂ (6%) level. The nitrogen in the medium was depleted within 72 h of incubation (Table 7). Although the biomass was increasing, the chlorophyll a content did not show any significant increase after 3 days of incubation under all conditions.

### Example 8

Algal biomass production in raceway ponds and lipid extraction: To assess the feasibility of producing biodiesel from mixed/wild native isolates of microalgae, the preliminary consortium was cultivated in treated wastewater in 4 raceway ponds of 950 L capacity, each supplemented with approximately 250 ppm nitrogen as sodium nitrate and 5-6% CO₂ air mixture bubbled through 2 air stones at a rate of 10 L min⁻¹. After 10 days, the biomass was harvested by centrifugation. Harvested algae with approximately 15% solids were dried at 60° C. for 24 h for extraction of lipids and biomass analysis. Lipids were extracted with hexane in a Soxhlet apparatus operated at 80° C. for 10 h after Miao & Wu ((2006) Bioresour. Technol. 97: 841-846). After Soxhlet extraction, hexane was evaporated using a rotary evaporator at 50° C. and 100 mbar to obtain lipids. TLC was performed to purify triglycerides (Touchstone J. C. (1995) J. Chromatogr. 671, 169-195). Fatty acids were methylated using the procedure described by Park & Goins ((1994) J. Food Sci. 59: 1262-1266) and run on a Supelcowax-10 wide bore capillary column in Shimadzu GC 14-A. Fatty acid peaks were identified against the chromatogram of a mixed fatty acid methyl ester standard (Nu-Chek Prep, Inc).
Biomass production in raceways, VTRs, and polybags: Different reactor configurations, raceway ponds, VTRs and polybags, were selected to assess the algal biomass production potential using carpet industry untreated wastewater. Atmospheric temperature increased from the first run to the last run, as did the light intensity. The rise in the mean diurnal temperature was from 11.1° C. to 21.1° C., while the mean insolation increased from 14.5 MJ m⁻² d⁻¹ to 17.9 MJ m⁻² d⁻¹ from the first run to the fourth.
Comparison of vertical tube reactors with raceways: In a run of 11 days, carpet industry untreated wastewater was used to compare the biomass productivity in raceways with that in VTRs. Raceways with 496 L of wastewater were inoculated with 18 L of each of the three algal cultures mentioned above and operated at 20 cm depth and a total volume of 550 L (Table 8). VTRs had 88 L wastewater and 4 L of each algal culture added as inoculum. VTRs were operated with 100 L working volume and the depth of water column was maintained at 61 cm (Table 8).

In another run, carpet industry untreated wastewater was used to compare biomass productivity in raceways (working volume, 500 L; depth, 18 cm), vertical tank reactors (working volume, 100 L; diameter, 45 cm; depth, 61 cm) and polybags (working volume, 20 L; diameter, 16 cm; depth, 95 cm). Inoculum included equal volumes of the algal consortium that included the three strains C. globusa, C. minutissima and S. bijuga (Table 8). To each of the raceways, VTRs and polybags, 450, 90 and 18 L, respectively, of carpet industry untreated wastewater was filled and 25, 5 and 1 L of the consortium was added, respectively. Final volumes were: 500 L (18 cm deep) in raceways, 100 L (61 cm deep) in VTRs and 20 L (95 cm deep) in polybags.

Algae grown in carpet industry untreated wastewater recorded a volumetric biomass productivity of 0.015 g L⁻¹ d⁻¹ in raceway ponds when the depth was maintained at 30 cm (Table 8).

TABLE 8
Volumetric and areal biomass productivity of algal
consortium in carpet industry untreated wastewater

| | | | | | | Biomass productivity | | | | |
|---|---|---|---|---|---|---|---|---|---|---|
| | | | Reactor surface area | | | Volumetric | | Areal | | Duration |
| | Depth^{a} | Vol | Footprint | Illuminated | S:V | g L⁻¹ d⁻¹ | | g m⁻² d⁻¹ | | of run |
| Reactor | (cm) | (m³) | (m²) | (m²) | (m⁻¹)^{b} | Mean | SD | Mean | SD | (days) |
| RW | 30 | 0.95 | 3.1 | 3.1 | 3.3 | 0.015 | 0.002 | 4.42 | 0.75 | 10 |
| RW | 30 | 0.95 | 3.1 | 3.1 | 3.3 | 0.021 | 0.001 | 6.43 | 0.02 | 12 |
| RW | 20 | 0.55 | 2.8 | 2.8 | 5.1 | 0.04 | 0.001 | 7.79 | 0.06 | 11 |
| RW | 20 | 0.55 | 2.8 | 2.8 | 5.1 | 0.036 | 0.002 | 7.13 | 0.34 | 11 |
| VTR | 45 | 0.1 | 0.16 | 1 | 10 | 0.032 | 0.002 | 20.3 | 1.04 | 11 |
| RW | 18 | 0.5 | 2.8 | 2.8 | 5.6 | 0.057 | 0.001 | 10.36 | 0.06 | 8 |
| RW | 18 | 0.5 | 2.8 | 2.8 | 5.6 | 0.045 | 0.001 | 8.04 | 0.1 | 8 |
| VTR | 45 | 0.1 | 0.16 | 1 | 10 | 0.044 | 0.011 | 27.4 | 6.57 | 8 |
| PB | 15 | 0.02 | 0.021 | 0.5 | 25 | 0.07 | 0.018 | 66.4 | 16.8 | 8 |

RW—Raceways; VTR—Vertical tank reactors; PB—Polybags;
^{a}Light penetration depth perpendicular to largest surface area;
^{b}S:V, Surface to Volume Ratio

A second run recorded a productivity of 0.021 g L⁻¹ d⁻¹ which was 40% higher than the first run. Average biomass productivity of the two raceways in the third run with 20 cm depth showed 153 and 81% increase when compared to the first and second runs, respectively. The raceway ponds maintained with 18 cm depth recorded a maximum average productivity of 0.051 g L⁻¹ d⁻¹ which was 3.4, 2.4 and 1.3 times higher than the first, second and third runs, respectively, as shown in Table 7.

Volumetric productivity of raceways was between about 19 and about 16% more than the productivity obtained in the VTRs. However, volumetric productivity with the polybags was 0.07 g L⁻¹ d⁻¹, which was significantly higher than the other two reactor systems (Table 8). Decrease in the depth of water from 30 cm to 20 cm in raceways enhanced volumetric productivity by 81%, whereas the areal productivity showed only a 16% increase. A further decrease in depth to 18 cm showed 34% and 23% increases in volumetric and areal productivities, respectively. In a run that made the direct comparison of all reactors, polybag reactors recorded between about 37% and about 59% increase in volumetric productivity, and between about 621% and 142% increase in areal productivity over raceways and VTRs, respectively (Table 8).

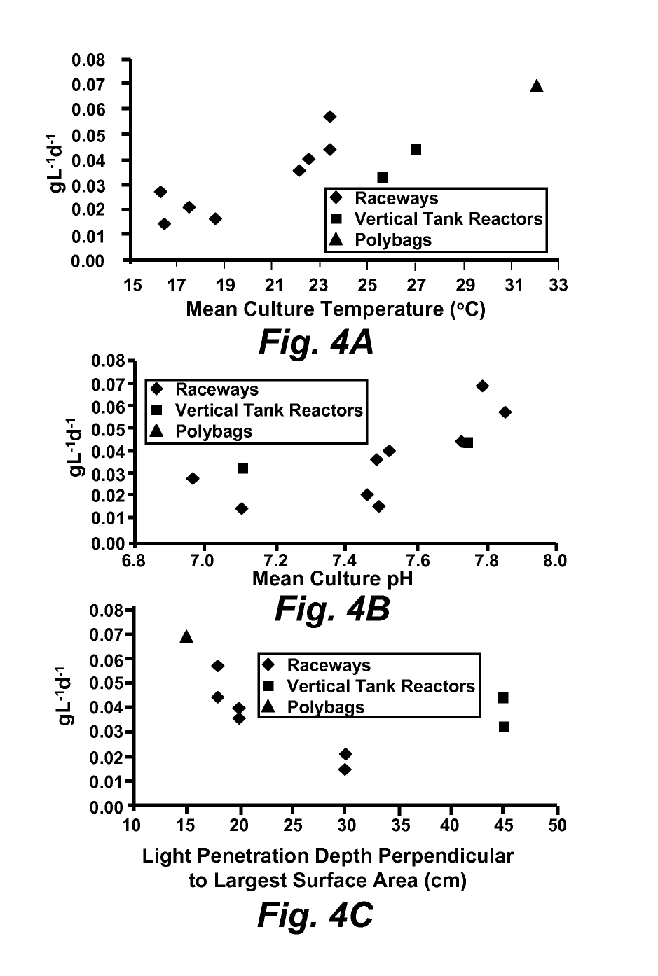

Changes in the temperature impacted biomass productivity. Greenhouse temperatures from early to late afternoon were 6° C. higher than ambient. Polybags recorded highest temperatures up to 43° C., and the broadest range of variation in diurnal temperature. A rise in temperature from 16° C. to 24° C. led to increases in productivity of the raceways, as shown in FIG. 4A. When the direct comparison was made between the three reactor types, the average temperatures were 24° C., 27.5° C. and 32.1° C. for the raceways, VTRs and polybags, respectively.

Compared to raceways, polybags recorded 8.1° C. increases and VTRs recorded 3.5° C. increases in the culture temperature. The polybags maintained a temperature that more favored higher biomass productivity (FIG. 4A). However, in contrast to the polybags, the average volumetric productivity obtained in the VTRs was less than that of raceways. Growth was also directly proportional to increase in pH from 7.0-7.9, as shown in FIG. 4B.

Variation in the nutrient quality of the wastewater could also have varied the productivity, as is evident from Table 4C. Carpet industry untreated wastewater used in this study was colored due to the use of dyes in the carpet manufacturing process. Although the potential toxicity of the carpet industry dyes toward the algae was unknown, the dyes present did not prevent an increase in algal biomass productivity in raceway ponds. Both the volumetric and areal productivities showed significant increases matching the overall improvement in sunlight availability and ambient temperature (Table 8). These improvements were achieved over periods ranging from 8-12 days. During the fourth run the raceway areal productivity was in the range of 8.04-10.36 g m⁻² d⁻¹. Thus, despite potential limitations due to carpet industry pigments, higher productivities can be achieved as sunlight intensity increases and temperatures improve (Table 8; FIG. 4C).

To assess the interaction between biomass, temperature, pH, light and light penetration depth, a correlation analysis was performed. Correlation with light was not statistically significant for any of the observed parameters (Table 9).

TABLE 9
Correlation analysis of interaction between biomass productivity,
temperature, pH and light penetration depth in raceways,
vertical tank reactors (VTRs), and polybags

| Parameter | Light | Temperature | pH | Depth^{b} | Biomass |
|---|---|---|---|---|---|
| Light | r² | 0.548 | 0.370 | −0.401 | 0.454 |
| | P | 0.065^{a} | 0.236^{a} | 0.196^{a} | 0.138^{a} |
| Temperature | r² | | 0.677 | −0.862 | 0.790 |
| | P | | 0.0156 | 0.0003 | 0.0022 |
| pH | r² | | | −0.609 | 0.926 |
| | P | | | 0.0357 | 0.00002 |
| Depth | r² | | | | −0.836 |
| | P | | | | 0.0007 |

^{a}No significant correlation between two variables if P value is ≧0.050;
^{b}Light penetration depth perpendicular to largest surface area for raceways, VTRs, and polybags

All others showed significant correlation with each other, indicating multi-collinearity amongst these variables (Table 8) that could act as predictors for the productivity. Therefore regression analyses were performed removing such factors, one at a time. The following equations were significant:

Biomass=−2.065+(0.305×pH) R²=0.857

Biomass=1.207−(0.0365×depth) R²=0.699

Biomass=−0.924+(0.218×pH)−(0.0189×depth) R²=0.975

Biomass=−1.823+(0.0123×Temperature)+(0.238×pH) R²=0.906

Biomass=0.706+(0.011×Temp)−(0.0264×depth) R²=0.718

Biomass=−0.768+(0.229×pH)−(0.0059×Temperature)−(0.0234×Depth) R²=0.980

Acidity (pH) was the most important factor, showing highly significant positive correlation with biomass productivity. It cannot be used to determine the optimum pH for the growth of algae since although algal growth is affected by pH, the later increases with growth of algae due to the consumption of carbon dioxide. However, it could be used as a good predictor for algal productivity.

Light penetration was the next most significant factor since greater culture depth results in more of the volume of the raceway not receiving sufficient light to support photosynthesis. All other equations with double and triple predictors as shown above had good predictability although multicollinearity can affect their usefulness as predictors.

In general, productivity of an algae cultivation system can be evaluated through the four parameters of volumetric productivity (VP), i.e. productivity per unit reactor volume (g L⁻¹ d⁻¹); illuminated surface productivity (ISP), i.e. productivity per unit of illuminated surface area of the reactor (g m⁻² d⁻¹); areal productivity (AP), i.e. productivity per unit of ground area occupied by the reactor (g m⁻² d⁻¹); and overall areal productivity (OAP) expressed as g m⁻² d⁻¹, i.e. the productivity obtained from the overall ground area including empty spaces required for equipment access and space between reactors in a mass cultivation system (Tredici M. R. (2004) in: Richmond A, ed: Handbook of Microalgae Culture: Biotechnology and Applied Phycology. Oxford, Blackwell Publishing, pp 178-214). OAP has greater meaning and provides a useful method to evaluate productivity between different kinds of cultivation systems and reactors for scale-up operations.

Table 10 provides the productivity comparison between 3 different reactor systems evaluated based on AP, OAP and ISP.

TABLE 10
Areal, overall and illuminated surface area productivity and photosynthetic efficiency
of algae cultivated in raceways, vertical tank reactors (VTRs), and polybags

| | | | | Photosynthetic | |
|---|---|---|---|---|---|
| | Productivity^{a} | | Mean Solar | efficiency based on | |
| | (g m⁻² d⁻¹) | | Radiation | full solar spectrum | Expected Yield |
| Reactor | Mean | SD | MJ m⁻² d⁻¹ | (%) | tons ha⁻¹ year⁻¹ |

Areal productivity (AP) based on actual footprint^{b}

| | | | | | |
|---|---|---|---|---|---|
| Raceways | 7.4 | 2.0 | 16.7 | 1.0 | 27.0 |
| VTRs | 23.9 | 5.0 | 20.2 | 2.6 | 87.2 |
| Polybags | 66.4 | 16.8 | 17.9 | 8.1 | 242.4 |

Overall areal productivity (OAP) based on system's estimated footprint^{c}

| | | | | | |
|---|---|---|---|---|---|
| Raceways | 5.9 | 1.6 | 16.7 | 0.8 | 21.5 |
| VTRs | 8.1 | 1.7 | 20.2 | 0.9 | 29.6 |
| Polybags | 21.1 | 5.4 | 17.9 | 2.6 | 77.0 |

Illuminated surface area productivity (ISP)^{d}

| | | | | | |
|---|---|---|---|---|---|
| Raceways | 7.4 | 2.0 | 16.7 | 1.0 | 27.0 |
| VTRs | 3.8 | 0.8 | 20.2 | 0.4 | — |
| Polybags | 2.8 | 0.4 | 17.9 | 0.3 | — |

^{a}Productivity represented in the table is an average of runs 1, 2, 3 and 4 for raceway and runs 3 and 4 for VTRs;
^{b}Areal productivity: Biomass (g) produced per unit area (m²) per unit time (day). Areal productivity = (B₂ − B₁)/A/(T₂ − T₁) where: B₂, biomass at time T₂; B₁, biomass at time T₁; and F, system's actual footprint area (m²). Actual footprint area for raceways, VTRs and polybags were 2.9, 0.16 and 0.021 m²;
^{c}Overall areal productivity based on system's estimated footprint was calculated based on 25% additional area required for raceways in addition to the actual footprint. For VTRs and polybags the estimated footprint was calculated based on the additional area required for operational convenience such as empty space between reactors and ground area to avoid shading effect for achieving optimum productivity per ha. Estimated foot print area for VTRs was 0.47 m² and polybags was 0.066 m^{2;}
^{d}ISP-calculated based on the illuminated surface area of 2.9, 1 and 0.5 m2 for raceways, VTRs and polybags, respectively.

Though the productivity trend observed for AP and OAP were same, where the polybags showed greater productivity followed by VTRs and raceways, ISP gave a reverse picture where raceways recorded higher productivity followed by VTRs and polybags. Accordingly, ISP is not suitable to evaluate vertical reactor systems for mass production since the illuminated surface area of VTRs was 6.25 times the occupied surface area; whereas it was 23 times for polybags (Table 8). For horizontal systems such as raceways, the illuminated surface area remained the same as occupied surface area. Thus, OAP was selected as the right parameter to avoid erroneous extrapolation based on AP and ISP.

Among all the reactors, polybags showed significant increase in OAP and AP followed by VTRs and raceways (Table 10). AP and OAP of polybags showed 9-fold and 3.6-fold increases over raceways, and 2.8-fold and 2.6-fold increases over VTRs, respectively; whereas VTRs showed 3.2 and 1.4 times increase over raceways for AP and OAP, respectively (Table 10). Thus, the higher AP was achieved by diluting the light energy over a larger bioreactor surface area of the cultures, taking advantage of the vertical height of the VTRs and polybag reactors, in agreement with the observations made by Lee Y. K. ((2001) J. Appl. Phycology 13: 307-315). Light dilution reduces the negative effects of photosaturation and photoinhibition, leading to significant increases in photosynthetic efficiency and productivity (Zitelli et al., (2006) Aquaculture 261: 932-943). The photosynthetic efficiency of polybags calculated based on AP and OAP was much higher (8.1 and 2.6%, respectively) than VTRs and raceways (Table 10).

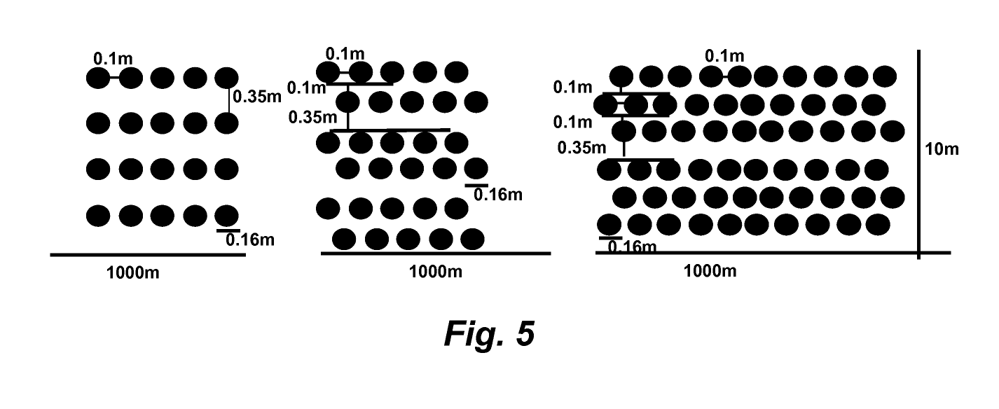

Various arrangements for polybag reactors were evaluated to obtain maximum productivity in large-scale production systems. To maximize the biomass productivity per unit area, polybag arrangements in a 1000 m×10 m plot in single row, paired rows and triple row cassettes were considered, as schematically shown in FIG. 5. Based on the assessment, it can be estimated that a maximum of about 50 and about 80 tons of biomass ha⁻¹ year⁻¹ can be obtained using triple row cassettes arrangement for 20 and 30 L capacity polybags, respectively (Table 11).

TABLE 11
Polybag arrangements for attaining maximum
biomass productivity in 20 L and 30 L bags

| | | | | Biomass | |
|---|---|---|---|---|---|
| | | | Total | productivity | |
| | Distance covered (m) by a set | | number | (tons ha⁻¹ year⁻¹) | |
| | of rows and columns | | of bags | Polybag capacity | |
| Arrangement | Row | Column | ha⁻¹ | 20 L | 30 L |
| Single foil | 0.16^{a} + 0.1^{b} | 0.16^{a} + 0.35^{c} | 67,805 | 35 | 52 |
| Paired rows | 0.16^{a} + 0.1^{b} | 2^{e} (0.16^{a}) + 0.1^{c} + 0.35^{d} | 89,820 | 46 | 69 |
| Triple cassettes | 0.16^{a} + 0.1^{b} | 3^{e} (0.16^{a}) + 2 (0.1)^{c} + 0.35^{d} | 100,721 | 51 | 77 |
| Compact | 0.16^{a} | 0.16^{a} | 497,611 | 254 | 381 |

^{a}Polybag diameter;
^{b}bag to bag distance in a row;
^{c}bag to bag distance in a column;
^{d}pair to pair or cassette to cassette distance;
^{e}No. of rows per pair or cassette;
*Calculations were based on 1 ha area with a dimension of 1000 m × 10 m

In this arrangement, the rows over the longer axis have a space of 0.1 m between bags and the columns over the smaller axis have cassettes of three rows with the same distance of polybags (0.1 m) but 0.35 m distance between two cassettes (Table 11, FIG. 5). Such an arrangement can accommodate 100,721 polybags. In contrast the maximum estimated OAP in raceways and VTRs was 21.5 and 29.6 tons ha⁻¹ year⁻¹, respectively (Table 10).

The performance of the closed systems was due to: (i) better temperature profile, i.e. the culture in the polybags and VTRs reached the optimal temperature for growth earlier in the day when compared to the raceway ponds. Reaching the optimal temperature in the early morning prolongs the duration of effective photosynthesis, which was not the case with raceways; (ii) higher surface to volume ratio. Based on the average values from Table 8, the surface to volume ratio of polybags was 25 m⁻¹ whereas it was only 10 m⁻¹ and 4.7 m⁻¹ for VTRs and raceways, respectively; and (iii) narrow light path: Lee Y. K. ((2001) J. Appl. Phycology 13: 307-315) reported that the narrow light path (1.2-12.3 cm) in enclosed tubular and flat plate bioreactors allows cell concentration to reach a higher value of up to approximately 20 g L⁻¹ and a volumetric biomass productivity of 0.25-3.64 g L¹ d⁻¹ in outdoor fed batch cultures. The light penetration depth perpendicular to the largest surface area in polybags was 15 cm, which resulted in higher volumetric productivity than VTRs (Table 8).

Unexpectedly, the volumetric productivities of VTRs were lower than raceways. Though the light receiving surface to volume ratio of VTRs was more than the raceway, the walls of the tubes caused a 30% decrease of sunlight penetration from the outer to the inner face of the walls. Significantly larger light penetration depth (approximately 2.5 times as deep for the tubes compared to the raceways) and the light attenuation of the vertical tank walls may have resulted in the poor volumetric productivity despite large surface to volume ratio, less variation in temperature and efficient CO₂ mass transfer conditions.

### Example 9

Biodiesel production from consortium: Biodiesel from crude microalgae oil was obtained by a two step process: acid trans-esterification followed by a base trans-esterification due to the high acid value of crude algal oil. The free fatty acids were converted into esters. The determination of the fatty acid profile was based on AOCS Method Ce 1c-89 using a PerkinElmer Inc. Clarus 600 GC-FID equipped with a Supelco SP 2340 fused silica column (Sigma-Aldrich Co.). The GC oven was heated to 150° C., ramped to 200° C. at 1.3° C. min⁻¹ and held at 200° C. for 20 mins. The helium flow was 2.0 mL min⁻¹ at 1.6 psi and the FID temperature was 210° C. Biodiesel was diluted to a 1% solution in heptane before injection. The core properties of biodiesel such as free glycerin and total bound glycerin were measured in a GC as per ASTM D-6584 (2004) test methods.
Algal oil characterization and biodiesel conversion: To demonstrate the feasibility of producing biodiesel from algal consortium grown in treated wastewater, about 126.7 g (144 mL) of crude algal oil was extracted from 2.3 kg of dry algal biomass. The energy content of the crude algal oil was 40.2 MJ kg⁻¹. A compositional analysis of crude algal oil is shown in Table 12. After conversion of the oil to methyl esters, the fatty acid profile was determined, as shown in Table 12.

TABLE 12
Fatty acid profiles of crude and purified algal oils and algal biodiesel

| | | |
|---|---|---|
| Crude | Refined | Algal |
| algal oil | algal oil | biodiesel |

| Fatty acids | | (%) | | |
|---|---|---|---|---|
| C14:0 | Myristic | 1.91 | 1.4 | 0.9 |
| C15:1 | Pentadecenoic | | 5.9 | |
| C16:0 | Palmitic | 20.62 | 17.6 | 16.3 |
| C16:1 | Palmitoleic | 6.47 | | 5.8 |
| C18:0 | Stearic | 1.43 | | 1.2 |
| C18:1 | Oleic | 10.58 | 14.9 | 12.1 |
| C18:2 | Linoleic | 10.54 | | 20 |
| C18:2n6 cis | Linoleic | | 9.6 | |
| C18:3 | Linoleic | | 11.8 | |
| | Linolenic | 15.47 | 38.8 | 27.9 |
| C20:0 | Arachidic | 0.04 | | |
| C20:1 | Gadoleic | 1.1 | | |
| C20:2 | Eicosadienoic | 1.05 | | |
| C20:3 | Mead | 1.05 | | |
| C20:4 | Arachidonic | 0.88 | | |
| C20:5 | Timnodonic | 1.48 | | |
| C22:0 | Behenic | 1.42 | | |
| C22:5 | Docosapentenoic | 0.41 | | |
| C22:6 | Docosahexenoic | 0.05 | | |
| Unknowns | | 25.5 | | 15.8 |
| Unsaturated^{a} | | 65.88 | 81 | 78.15 |
| Saturated^{a} | | 34.12 | 19 | 21.85 |

^{a}Percentage calculated based on the total known fatty acids

The purified fraction of triglycerides contained fatty acids ranging from C14:0 to C18:3 (Table 12). Both the crude algal oil and purified fraction of triglycerides were dominated by the presence of C16:0 (palmitic), C18:1 (oleic), and C18:3 (linolenic) acids (Table 12). Crude oil further contained C18:2 (linoleic), whereas the purified fraction showed the presence of cis and trans isomers of C18:2. Both the crude and purified oils were mainly composed of unsaturated fatty acids ranging from approximately 66 to approximately 81% among the known total fatty acids (Table 12), in conformity with the observations made by Gouveia & Oliveira ((2009) J. Ind. Microbiol. Biotechnol. 36: 269-274, that microalgal lipids derived from Chlorella vulgaris, Spirulina maxima, Nannochloropsis oleabundans, Scenedesmus obliquus and Dunaliella tertiolecta were mainly composed of 50-65% unsaturated fatty acids.

To determine if biodiesel can be produced from mixed cultures of native (wild) strains growing in the carpet industry treated wastewater, conversion of extracted crude algal oil to biodiesel was examined. The crude algal oil showed very high acid value approximately 99 (mg KOH g⁻¹ indicating about 50% free fatty acids, an undesirable trait for biodiesel conversion process. An acid catalyzed trans-esterification process is normally used for feedstocks containing high free fatty acid content (Xu et al., (2006) J. Biotechnol. 126: 499-507). The biodiesel conversion process was carried out without degumming and chlorophyll removal. The free fatty acids present in the oil were converted into methyl esters. The completion of the reaction was verified by the disappearance of the free fatty acid absorbances in FTIR spectrum. The estimate of conversion was greater than 95%, with a product yield on the acid esterification of about 70.9%. Losses were mainly due to oil impurities, soaps in oil and a small volume adhering to glass surface area.

The ASTM specification requires that the total glycerol and free glycerin be less than 0.24 and 0.02% of the final biodiesel product, respectively as measured using a gas chromatographic method described in ASTM D 6584. The biodiesel made from mixed algal biomass was found to contain 0.0155 and 0.0001% total bound and free glycerin, respectively, meeting the ASTM specifications. This was further confirmed with a GC analysis and the Near Infrared Spectroscopy. The yield of biodiesel from starting oil after the base transesterification was 63.9%. However the final recovery of methyl esters was only 38.7% due to various losses in the base transesterification and purification. For biodiesel, the FTIR spectra was characterized by a series of peaks from 3100 cm⁻¹ to 2750 cm⁻¹, a strong peak from 1745 cm⁻¹ to 1740 cm⁻¹, a series of peaks from 1470 cm⁻¹ to 1430 cm⁻¹, a peak at 1360 cm⁻¹, as well as a series of peaks from 1220 cm⁻¹ to 1160 cm⁻¹, 1020 cm⁻¹ to 970 cm⁻¹, 920 cm⁻¹ to 840 cm⁻¹, and a peak at 720 cm⁻¹. These peaks were characteristic of the long-chain fatty acid methyl esters predominant in biodiesel. The ester FTIR showed primarily methyl esters, no free fatty acid, and no soap. Algal methyl esters were predominated by C18:3 (linolenic), C18:2 (linoleic), C16:0 (palmitic), C18:1 (oleic) and C16:1 (palmitoleic) (Table 12). Unsaturated fatty acids in algal biodiesel constituted approximately 65.8% of the known total fatty acid fraction. EN 14214 (2004) specifies a limit of 12% for linolenic (C18:3) acid, for a quality biodiesel, whereas the biodiesel produced from algal consortium showed 27.9% of C18:3. It is contemplated, however, that the quality of biodiesel can be improved if blended with other sources of biodiesel derived from non-food feedstocks.

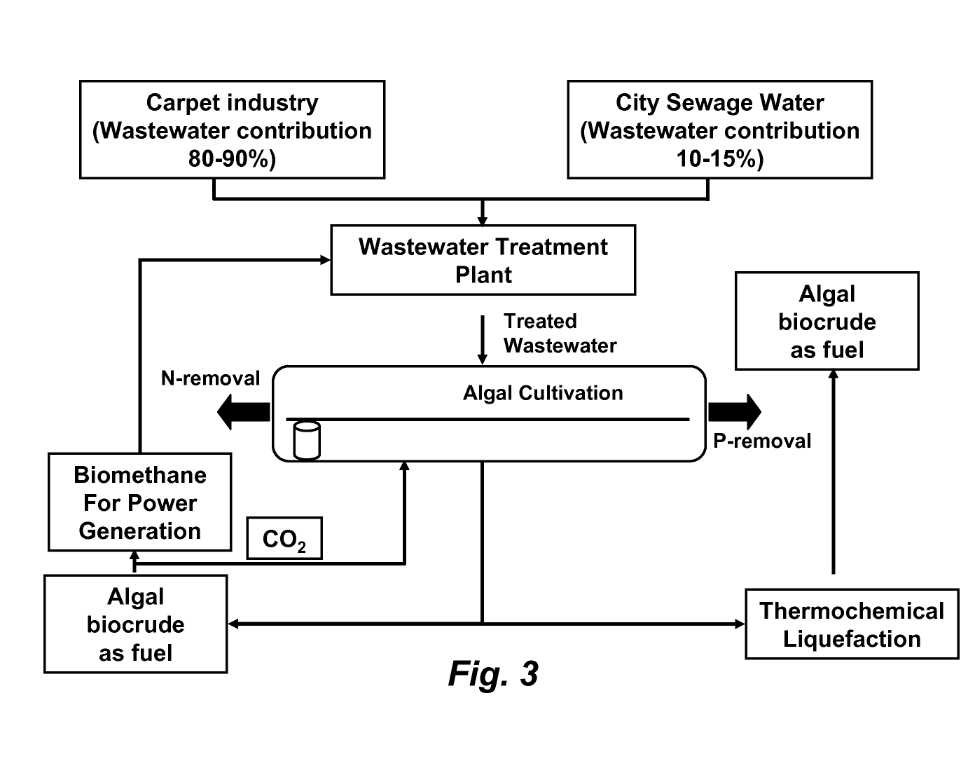

These results indicate that the algal oil produced from mixed cultures of native algae can be used for biodiesel production. This is the first report on production of biodiesel from a native algal consortium using treated carpet industrial wastewater containing 10-15% sewage mix. Though the lipid content of this consortium was very low, the energy stored in the biomass could be also recovered through thermochemical liquefaction where the algal biomass can be converted directly to a biocrude with a recovery rate of 30-44% and a heating value of 34.7 KJ g⁻¹ (Amin S. (2009) Energy Convers. Manage. 50: 1834-1840), or into biogas through anaerobic digestion. An alternate scheme for biofuel production using carpet industry wastewater is presented in FIG. 3.

### Example 10

Quantification of pigments and other parameters: After harvesting 10 mL of homogenized algal cells by centrifugation (5000 rpm, 10 min), the algal pellet was exhaustively extracted with hot methanol until it was colourless. Chlorophyll (chl) a concentration was spectrophotometrically determined with the extinction coefficients in methanol and calculated after Porra et al. (1989).

To determine biomass, 4.7 cm Whatman GF/C glass fibre filters were dried at 90° C. for 4 h, vacuum desiccated to cool to room temperature and weighed. Biomass was determined by filtering 10 mL of culture which was passed through these preweighed filters, washed with 10 mL of 0.65 M ammonium formate solution to remove excess salts and dried and weighed as above.

Lipid content was measured gravimetrically with Ankom XT10 automated extraction system using hexane as solvent. Algal culture was filtered through a preweighed 4.7 cm Whatman glass fiber filter and washed with ammonium formate and deionized water to remove any salt residues. The filters were dried at 60° C. overnight in a forced-air oven and cooled in a desiccator. They were weighed (W₁), inserted into Ankom XT4 extraction bags and sealed. After drying, the extraction bags were kept in a resealable plastic bag with desiccant material while each individual bag was removed and weighed (W₂). The extraction bags were then placed into the extractor and the extraction was performed for 1 h at 90° C. with hexane as solvent. After extraction, the bags were then transferred to the forced-air oven and dried at 60° C. overnight and cooled in a desiccator. The bags were reweighed (W₃) and the following formula was used to calculate the lipid content of the algae samples:

Lipid %=(W₂−W₃)/W₁×100

Nutrient Analysis was done using the automated cadmium reduction method, ascorbic acid reduction method and the phenate method for the determination of nitrate, phosphate and ammonium, respectively. Total nitrogen and total phosphorus were determined using the persulfate method which uses simultaneous digestion of nitrogen and phosphorus components. Analysis of other parameters for wastewater was done as per the standard procedures (APHA-AWA-WEF, 2005).

### Example 11

Algal biomass production in raceway ponds: Each batch was cultivated for 10-12 days. The average productivity observed in winter was 2.64 g m⁻² d⁻¹ or 9.3 tons of dry biomass ha⁻¹ year⁻¹ with maximum biomass productivity being 4.9 g m⁻² d⁻¹ or 17.8 tons ha⁻¹ year⁻¹. The consortium showed remarkable resistance to predation and crash and exhibited tolerance to low temperatures.

Biomass obtained from algal consortium grown in raceways was analysed for its composition before and after lipid extraction, as shown in Table 13.

TABLE 13
Compositional analysis of the algal consortium
before and after lipid extraction

| | | |
|---|---|---|
| | Biomass before | Biomass after lipid |
| Parameters | lipid extraction | extraction |

Proximate analysis (%)

| | | |
|---|---|---|
| Moisture | 7.59 ± 0.16 | 6.44 ± 0.73 |
| Volatiles | 68.89 ± 0.15 | 67.33 ± 0.86 |
| Ashes | 11.42 ± 0.11 | 13.39 ± 1.76 |
| Fixed carbon | 12.10 ± 0.120 | 12.82 ± 0.20 |

Ultimate analysis (%)

| | | |
|---|---|---|
| Carbon (C) | 49.44 ± 0.11 | 45.95 ± 1.08 |
| Hydrogen (H) | 6.65 ± 0.03 | 6.16 ± 0.13 |
| Nitrogen (N) | 9.27 ± 0.18 | 9.28 ± 0.88 |
| Sulfur (S) | 0.67 ± 0.03 | 0.76 ± 0.10 |
| Oxygen (O) | 21.62 ± 0.27 | 23.55 ± 0.35 |
| Higher heating value, (HHV) | 22.87 ± 0.51 | 20.77 ± 0.42 |
| (MJ kg⁻¹) | | |

Biochemical composition (%)

| | | |
|---|---|---|
| Protein | 54.50 ± 0.40 | 56.9 ± 4.20 |
| Lipids | 6.82 ± 0.08 | 0.4 ± 0.06 |
| Carbohydrates | 8.98 ± 0.87 | 9.15 |
| Phosphorus | 0.87 | 1.4 |

The recovered oil was dark green in colour and contained gums, pigments, and the like.

Energy stored in the mixed algal consortium before and after lipid extraction was 22.87 MJ kg⁻¹ and 20.77 MJ kg⁻¹, comparable to previously determined values for algae (Huntley & Redalje, (2006) Mitig. Adapt. Strategies Glob. Chang. 12: 573-608; Sheenan et al., (1998) NREL Report No. TP-580-24190). A 9% reduction in the energy value of fresh algal biomass was observed after lipid extraction. The C:N:P ratio of the algal consortium before and after lipid extraction was 57:11:1 and 33:7:1, respectively. The biomass carbon content showed a drastic decrease due to lipid extraction.

The algal consortium was rich in proteins (approximately 54.5%) and low in lipids and carbohydrates, as shown in Table 12, possibly due to the dominance of protein-rich strains like Scenedesmus in the consortium. Though this study observed low lipid content in the consortium, it is contemplated that the energy present in the algal biomass can also be recovered through anaerobic digestion via biomethane.

### Example 12

Statistical analysis: In case of carpet industry untreated wastewater, the productivity was normalized by deducting the zero day observations of biomass from the final day. The data set were then used to form zero order correlation matrix vis-á-vis daily irradiation, temperature, pH, light penetration depth and biomass. Multicollinearity was determined and based on the data, regression analysis was performed to determine the best predictor parameter.

### Example 13

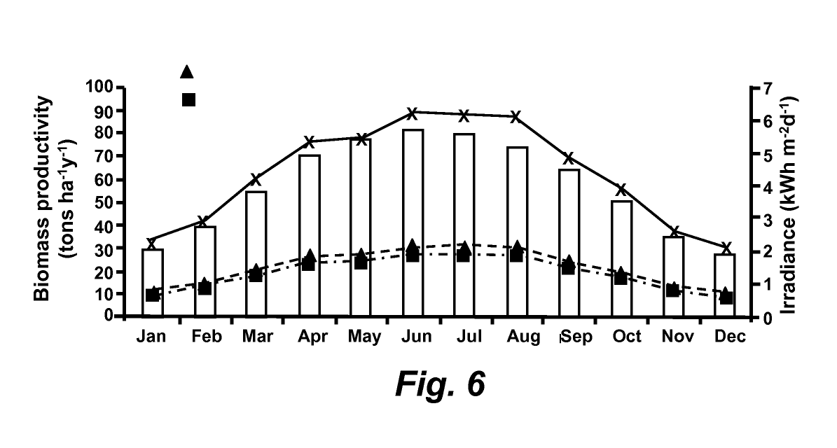

Estimated biomass productivity for a carpet industry dependent city: As shown in FIG. 6, an analysis was done to estimate the biomass productivity for an area having more than 150 carpet mills in north Georgia, U.S.A. using a raceway, a VTR, or a polybag reactor based on 22 year weather data of NASA Langley Research Center Atmospheric Science Data Center (New et al., (2002) Clim. Res. 21: 1-25). The average irradiation year⁻¹ for the area is 4.02 kWh m⁻² d⁻¹ (167 W m⁻² s⁻¹ or 14.4 MJ m⁻² d⁻¹) and therefore it was estimated that a maximum biomass productivity of 28, 31 and 90 tons ha⁻¹ year⁻¹ could be obtained for raceways, VTRs and polybags, respectively during June and a minimum biomass productivity of 9, 10 and 30 tons ha⁻¹ year⁻¹ during December (FIG. 6). Average annual biomass productivity was estimated as 19, 22 and 62 tons ha⁻¹ year⁻¹ for raceways, vertical tank reactors and polybags, respectively.

### Example 14

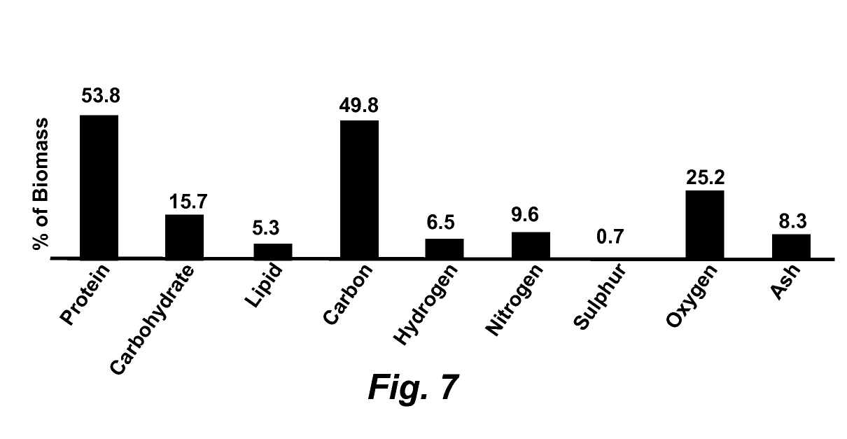

Biomass analysis of algal consortium: To assess the suitability of the biomass derived from algal consortium grown in carpet industry untreated wastewater, biomass characterization was done including proximate and ultimate analysis (FIG. 7). The mean carbon content of all harvested biomass was 49.8% whereas the mean nitrogen content was 9.6% (FIG. 7). This narrow C/N ratio of 5.2 suggested that a large percentage of the biomass was protein (approximately 53.8%). The hexane extracted neutral lipids were only 5.3% and the total carbohydrate was approximately 15.7%. However, the harvested biomass does possess a significant amount of energy per unit mass. The observed calorific value of 23.6 KJ g⁻¹ for the mixed algal biomass was within the values cited in other literature which range from 20 to 25 kJ g⁻¹ (Acièn Fernández et al., (1998) Biotechnol. Bioeng. 58: 605-616; Huntley & Redalje, (2006) Mitig. Adapt. Strategies Glob. Chang. 12: 573-608; Sheehan et al., (1998) NREL Report No. TP-580-24190).

### Example 15

Wastewater grown algae as energy crop: The potential of wastewater grown algae as an energy crop for biomethane production was assessed (Table 14).

TABLE 14
Biomethane and bioenergy production potential of energy
crops and algae grown in carpet industry wastewater

| | | | | Estimated energy | Estimated energy |
|---|---|---|---|---|---|
| | Biomass yield | Biomethane | Energy yield | recovered through | recovered through |
| | tons of | potential | in biomass | biomethane | biomethane^{e} |
| Biomass sources | VS ha⁻¹ | m³ ha⁻¹ y⁻¹ | GJ ha⁻¹ y⁻¹ | GJ ha⁻¹ y⁻¹ | kWh ha⁻¹ y⁻¹ |
| Maize^{a} | 15 | 8,850 | 265 | 352 | 97,891 |
| Cereals^{a} | 5 | 3,850 | 81 | 153 | 42,585 |
| Sunflower^{a} | 11 | 3,575 | 193 | 142 | 39,543 |
| Algae cultivated in | | | | | 134,144 |
| polybags | 58^{b} | 12,128^{c} | 1,265 | 483 | |
| Algae cultivated in | | | | | 51,567 |
| VTRs | 22^{b} | 4,662^{c} | 486 | 186 | |
| Algae cultivated in | | | | | 37,456 |
| raceways | 16^{b} | 3,386^{c} | 353 | 135 | |

^{a}Data on biomass yield and biomethane potential of maize, cereals and sunflower were adopted from Amon et al., (2007) Bioresource Technol. 98: 3204-3212;
^{b}Volatile solids (VS) constitute approximately 75% dry biomass of algal consortium;
^{c}Methane production from algal biomass was calculated based on 70% conversion of volatile solids and 0.3 m³ of methane production kg⁻¹ of VS; The values used for maize, cereals and sunflower were the average values calculated from the upper and lower ranges of Amon et al., (2007) Bioresource Technol. 98: 3204-3212;
^{d}Energy yield per ha was calculated based on the calorific value of 17.5 MJ kg⁻¹ for agricultural residues derived from maize, cereals and sunflower and 21.9 MJ kg⁻¹ for algae biomass;
^{e}Energy potential was calculated from the energy value of biomethane i.e. 0.0398 GJ m⁻³

Methane yield of algae biomass varies from 0.09 to 0.45 m³ kg⁻¹ of VS (Sialve et al., 2009). It has been reported that energy crops such as maize, cereals and sunflower could produce from about 2,600-10,200 m³ ha⁻¹ y⁻¹ of biomethane, respectively; whereas algae cultivated in carpet industry untreated wastewater in polybags could produce 12,128 m³ ha⁻¹ year⁻¹ of biomethane (Table 14). Estimated yields of biomethane and energy recovery from algae cultivated using carpet industry untreated wastewater was greater than the yields estimated for cereals and sunflower. Estimated energy recovered through biomethane from algae produced in polybags using carpet wastewater showed 37%, 215%, and 239% increases over the estimated biomethane energy recovered through maize, cereals and sunflower, respectively (Table 13). Algae produced in raceway showed the lowest estimated biomethane energy recovered per ha per year when compared to polybags and VTRs. This study estimated that the consortium of algae cultivated in polybags using carpet industry untreated wastewater could produce approximately 134 kWh of renewable power per hectare per annum.

What is claimed:

1. A method of generating an algal biomass, comprising:
(a) forming an algal culture by combining:
(i) a population of algal cells characterized as proliferating in a culture medium comprising carpet industry wastewater, and
(ii) a culture medium comprising carpet industry wastewater and a sewage system effluent; and
(b) maintaining the algal culture under conditions suitable for the proliferation of the population of algal cells, thereby forming an algal biomass.

2. The method of claim 1, wherein the medium, before receiving the population of algal cells is treated in a wastewater treatment plant.

3. The method according to claim 1, wherein the population of algal cells comprises at least one of the group consisting of: a marine algal strain, a freshwater (non-marine) algal strain, a cyanobacter strain, a diatomaceous algal strain, a plurality of marine algal strains, a plurality of freshwater (non-marine) algal strains, a plurality of cyanobacter strains, and a plurality of diatomaceous algal strains, or any combination thereof.

4. The method of claim 1, wherein at least one algal strain of the population of algal cells is isolated from a source in contact with the wastewater effluent of the carpet industry.

5. The method according to claim 1, wherein the population of algal cells comprises an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

6. The method of claim 4, wherein at least one algal strain of the population of algal cells is selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

7. The method of claim 1, wherein the population of algal cells comprises at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

8. The method of claim 6, wherein the population of algal cells consists of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

9. The method of claim 1, wherein the population of algal cells is a consortium, wherein the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, and a Chlamydomonas spp.

10. The method of claim 1, wherein the population of algal cells wherein the population of algal cells is a consortium comprising Chlamydomonas globosa, Chlorella minutissima, and Scenedesmus bijuga.

11. The method of claim 1, wherein the algal culture is contained within a raceway, a vertical tower reactor, or a polybag, and wherein the algal culture is optionally provided with air supplemented with carbon dioxide.

12. The method of claim 1, further comprising isolating the algal biomass from the medium.

13. A method of producing a biofuel from carpet industry wastewater comprising:
(a) forming an algal culture by combining:
(i) a population of algal cells characterized as proliferating in a medium comprising carpet industry wastewater, and
(ii) a culture medium comprising carpet industry wastewater and a sewage system effluent;
(b) maintaining the algal culture under conditions suitable for proliferation of the population of algal cells, thereby forming an algal biomass;
(c) isolating the algal biomass from the medium; and
(d) obtaining from the isolated algal biomass a biofuel or a source of a biofuel, wherein the step of obtaining from the isolated biomass a biofuel comprises the steps of isolating a lipid material from the biomass or converting the biomass to a biofuel, and wherein the isolated lipid material is converted to a biofuel.

14. The method of claim 13, wherein the medium, before receiving the population of algal cells is treated in a wastewater treatment plant.

15. The method according to claim 13, wherein the population of algal cells comprises at least one of the group consisting of: a marine algal strain, a freshwater (non-marine) algal strain, a cyanobacter strain, a diatomaceous algal strain, a plurality of marine algal strains, a plurality of freshwater (non-marine) algal strains, a plurality of cyanobacter strains, a plurality of diatomaceous algal strains, or any combination thereof.

16. The method of claim 13, wherein at least one algal strain of the population of algal cells is isolated from a source in contact with the wastewater effluent of the carpet industry.

17. The method of claim 13, wherein the population of algal cells comprises an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

18. The method of claim 16, wherein at least one algal strain of the population of algal cells is selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

19. The method according to claim 13, wherein the population of algal cells comprises at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

20. The method of claim 19 wherein the population of algal cells comprises a plurality of strains selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

21. The method of claim 13, wherein the population of algal cells is a consortium, wherein the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, and a Chlamydomonas spp.

22. The method of claim 13, wherein the population of algal cells wherein the population of algal cells is a consortium comprising Chlamydomonas globosa, Chlorella minutissima, and Scenedesmus bijuga.

23. The method of claim 13, wherein the algal culture is contained within a raceway, a vertical tower reactor, or a polybag, and wherein the algal culture is optionally provided with air supplemented with carbon dioxide.

24. A system for generating an algal biomass, the system comprising an algal culture container selected from a raceway, a vertical tower reactor, a polybag, or a plurality of any thereof, and wherein the container or plurality of containers is optionally provided with an air supply supplemented with carbon dioxide; an algal culture medium comprising carpet industry wastewater and optionally a sewage system effluent; and a population of algal cells in the algal culture medium, wherein the algal cells are selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

25. The system of claim 24, wherein the population of algal cells comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, and a Chlamydomonas spp.

26. The system of claim 24, wherein the system comprises a plurality of polybags.

27. An isolated population of algal cells comprising at least one algal strain isolated from a source in contact with the wastewater effluent of the carpet industry and capable of proliferating on a medium comprising carpet industry wastewater.

28. The isolated population of algal cells of claim 27, wherein at least one algal strain of the population of algal cells is selected from the group consisting of: a Chlamydomonas sp., Chlorella vulgaris, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

29. The isolated population of algal cells of claim 27, wherein the population of algal cells comprises an algal strain of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and a combination thereof.

30. The isolated population of algal cells of claim 27, wherein the algal population comprises at least one species selected from the group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, Pleurochrysis carterae CCMP 647, and a combination thereof.

31. The isolated population of algal cells of claim 27 wherein the algal population comprises a plurality of strains selected from the Group consisting of: Botryococcus braunii UTEX 572, Chlorella protothecoides UTEX 25, Chlorella saccharophila var. saccharophila UTEX 2469, Chlorella vulgaris UTEX 2714, Cricosphaera carterae UTEX LB1014, Dunaliella tertiolecta UTEX LB999, Nannochloris oculata UTEX LB1998, Spirulina platensis UTEX LB1926, Spirulina maxima UTEX LB2342, Tetraselmis suecica UTEX LB2286, Tetraselmis chuii UTEX LB232, Phaeodactylum tricornutum UTEX 646, and Pleurochrysis carterae CCMP 647.

32. The method of claim 27, wherein the population of algal cells is a consortium, wherein the consortium comprises Gleocytis vesiculosa strain 1, Limnothrix redekei, Gleocytis vesiculosa strain 2, Scenedesmus spp., Limnothrix redekei, Chlorococcum humicola strain 1, Chlorococcum humicola strain 2, Chlorococcum humicola strain 3, Clorella vulgaris strain 1, Clorella vulgaris strain 2, Clorella vulgaris strain 3, Gleocytis vesiculosa strain 3, Anabaena spp., Gleocytis vesiculosa strain 4, and a Chlamydomonas spp.

33. The isolated population of algal cells of claim 27, wherein the population of algal cells is a consortium comprising Chlamydomonas globosa, Chlorella minutissima, and Scenedesmus bijuga.
